(19) United States
(12) Patent Application Publication
(10) Pub. No.: US 2018/0014780 A1
(43) Pub. Date: Jan. 18, 2018

(54) CONDUCTIVE POLYMER ELECTRODES, WIRING ELEMENTS, AND USE THEREOF IN HEALTH AND SPORTS MONITORING

(71) Applicant: University of Connecticut, Farmington, CT (US)

(72) Inventors: Gregory Allen Sotzing, Storrs, CT (US); Ki Chon, Mansfield, CT (US)

(21) Appl. No.: 15/650,308

(22) Filed: Jul. 14, 2017

Related U.S. Application Data
(60) Provisional application No. 62/362,616, filed on Jul. 15, 2016.

(51) Int. Cl. A61B 5/00; A61B 5/0478; A61B 5/08; A61B 5/0492; A61B 5/0408; A61B 5/024; B05D 3/14

(52) U.S. Cl. CPC A61B 5/6804; A61B 5/6803; A61B 5/0408; A61B 5/0478; A61B 5/7203; A61B 5/6807; A61B 5/02438; A61B 5/0816; A61B 5/0492; A61B 2562/0209; A61B 2562/125; B05D 3/144

(57) ABSTRACT

Disclosed herein are conductive polymer electrodes and wiring elements for use as the conductive element in health monitoring applications, and more specifically to conductive polymer fabric electrodes or conductive polymer fabric wiring elements for use as the conductive element in pads for health monitoring applications and other wearable monitoring systems.

### CROSS-REFERENCE TO RELATED APPLICATION

This application claims the benefit of U.S. Provisional Application No. 62/362,616 filed Jul. 15, 2016, which is hereby incorporated by reference in its entirety.

### BACKGROUND

Electrodes play a key role in measurement and performance of vital signs monitoring devices. The most common electrocardiogram (ECG) electrodes are hydrogel silver/silver chloride (Ag/AgCl) electrodes. Typically, ECG electrodes are placed on the chest and limbs to measure the potential differences in the body that exist due to the varying electric field on the epidermis of skin. Traditionally, Ag/AgCl electrodes are used for ECG monitoring, accompanied by a hydrogel containing KCl electrolyte that reduces the impedance between the skin and electrode. Additionally, adhesive around Ag/AgCl on the electrode helps establish better skin contact and lower motion artifacts. Efforts have been made to integrate Ag/AgCl into textiles, but these electrodes suffer from poor signal due to high contact impedances. Also, Ag/AgCl has been shown to cause skin irritation during prolonged usage and drying of the hydrogel leads to unreliable ECG data.

Furthermore, although Ag/AgCl electrode usefulness and effectiveness in clinical environments has been established, their usability is compromised in wet conditions. Hydrophobic electrodes should capture all ECG morphological waveforms not only in dry condition, but also in different water compositions where fresh/unfiltered, chlorinated, and salt water are the more relevant types. Each water type has a different conductance due to varying ionic compositions where salt water poses the more challenging environment for ECG recording as the resistance can be as low as 10Ω for salty water. When exposed to highly ionic environments the impedance between the electrode and skin surface interface is significantly reduced. Therefore, an electrode designed for underwater ECG monitoring would have to be sufficiently isolated from the ionic components of the surrounding environment to function properly. In addition, an area of the individual's skin needs to be shaven in order to apply the Ag/AgCl electrode for ECG measurement. Often, due to skin irritation, the Ag/AgCl electrode cannot be attached to the same spot of the individual if a second, near term ECG measurement needs to be made. Furthermore, factors like limited shelf stability, disposability, and signal degradation over time, limit the use of Ag/AgCl hydrogel electrodes in environments outside of the clinical setting.

Furthermore, Ag/AgCl electrodes are not used for sports monitoring performance due to the unwillingness of the sporting goods industry to use metal in a monitoring device.

Comfort is a highly desirable feature of any monitoring device used in healthcare, sports medicine, and sports performance applications.

There remains a need in the art for new non-metal conductive materials that can be used in wearable monitoring devices to measure bio-potentials on the surface of living tissue.

### BRIEF SUMMARY

In one embodiment, an electrode or wiring element for use in a bio-potential wearable monitoring system comprises a conductive element embedded in a composite material; wherein the conductive element is a conductive polymer coated fabric.

In another embodiment, a method of making an electrode or wiring element for use in a bio-potential wearable monitoring system comprises embedding a conductive element in a composite material; wherein the conductive element is a conductive polymer fabric.

In an embodiment, a bio-potential wearable monitoring system comprises an electrode comprising a conductive polymer fabric, and a wiring element in electrical communication with the electrode; wherein the conductive polymer fabric is a.) an electrically conductive fibrous substrate comprising a fibrous substrate comprising polymeric fibers comprising nucleophile derivatized nanoparticles wherein a portion of the nucleophile derivatized nanoparticles are located at the surface of the polymeric fiber; and an electrically conductive polymer film disposed on at least a portion of the polymeric fibers of the fibrous substrate and at least in partial contact with the nucleophile derivatized nanoparticles; or b.) a stretchable electrically conductive structure comprising a stretchable insulating substrate comprising nucleophile derivatized nanoparticles located at the surface of the stretchable insulating substrate, wherein the stretchable insulating substrate is a fiber or fabric; and a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate.

In another embodiment, a method of measuring a bio-potential comprises using a monitoring system described herein.

In yet another embodiment, a method of making a bio-potential wearable monitoring system comprising forming an electrode comprising a conductive polymer fabric, and connecting a wiring element to the electrode, wherein the wiring element is in electrical communication with the electrode; and wherein the conductive polymer fabric is a.) an electrically conductive fibrous substrate comprising a fibrous substrate comprising polymeric fibers comprising nucleophile derivatized nanoparticles wherein a portion of the nucleophile derivatized nanoparticles are located at the surface of the polymeric fiber; and an electrically conductive polymer film disposed on at least a portion of the polymeric fibers of the fibrous substrate and at least in partial contact with the nucleophile derivatized nanoparticles; or b.) a stretchable electrically conductive structure comprising a stretchable insulating substrate comprising nucleophile derivatized nanoparticles located at the surface of the stretchable insulating substrate, wherein the stretchable insulating substrate is a fiber or fabric; and a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate.

### BRIEF DESCRIPTION OF THE DRAWINGS

FIG. 1 is an illustration of the electrode fabrication cavity mold and fabrication procedure showing the location of the copper mesh of a Comparative electrode.

FIG. 2 is a schematic view of a synthetic leather PET fiber.

FIG. 3 illustrates the ohmic behavior of an electrically conductive synthetic leather.

FIG. 4 illustrates the temperature dependent behavior of an electrically conductive synthetic leather: semiconductive below 0° C. and metallic above 0° C.

FIG. 5A and FIG. 5B illustrate the difference between synthetic leather with (Column B) and without (Column A) desiccant particles and PEDOT coated substrates prepared therefrom.

FIG. 6 is a schematic of the proposed phase segregation of PEDOT:PSS based on chemical reaction of the PSS with silica nanoparticles at the surface of a PET substrate.

FIG. 7 contains XPS results for PEDOT-PSS films on various substrates containing nucleophile derivatized nanoparticles (silica) compared to a control substrate free of nucleophile derivatized nanoparticles.

FIG. 8 Resistance versus temperature plot of: 4 wt. % PEDOT-PSS doped fabric (top graph); 7 wt % graphene/graphite infused fabric (middle graph); and 4 wt % PEDOT:PSS doped fabric containing 7 wt % graphene/graphite (bottom graph); the grey box insets of the top and middle graphs shows metallic behavior on a stretchable spandex substrate.

FIG. 9 Sheet resistance versus soak plot for samples having a 1 mm width line of PEDOT-PSS, where the samples have undergone a washing treatment.

FIG. 10 Sheet resistance versus soak plot for samples having a 1 mm width line of PEDOT-PSS and Scotchgard™ treatment, where the samples have undergone a washing treatment.

FIG. 11A is an illustration of ECG electrodes printed onto a compression t-shirt.

FIG. 11B is an illustration of a compression t-shirt comprising screen printed electrodes.

FIG. 12 ECG waveform obtained from PEDOT:PSS electrodes on t-shirt (-) and Ag/AgCl electrode ( - - - ) under different running speeds: FIG. 12(1) 80 bpm, FIG. 12(2) 110 bpm, FIG. 12(3) 140 bpm, FIG. 12(4) 150 bpm, FIG. 12(5) 180 bpm, and FIG. 12(6) 120 bpm.

FIG. 13 Electrochemical Impedance Spectroscopy of different electrodes: PEDOT:PSS electrode in Dry Condition (), PEDOT:PSS electrode in wet condition (▪), Ag/AgCl with Hydrogel (▴).

FIG. 14 shows the change in sheet resistance of a conductive polymer fabric as a function of wire thickness.

FIG. 15 illustrates an overlay of ECG signals obtained from two different electrode-wire configurations with Ag/AgCl electrodes.

FIG. 16 illustrates the variation in skin contact impedance of different electrodes: PEDOT:PSS electrode in dry conditions (A), commercially available Ag/AgCl electrode (B), PEDOT:PSS fabric electrode with lotion on skin (C).

FIG. 17 illustrates ECG responses of different electrodes under different skin conditions a subject: PEDOT:PSS electrode in dry conditions (A), PEDOT:PSS electrode with lotion on skin (B), Ag/AgCl (C) at rest.

FIG. 18 illustrates an ECG response from a screen printed PEDOT:PSS electrode with PEDOT:PSS wire with lotion on skin.

### DETAILED DESCRIPTION

Disclosed herein are conductive polymer electrodes and/or wiring elements for use as the conductive element in health monitoring and diagnostic applications, and more specifically to conductive polymer fabric electrodes or conductive polymer fabric wiring elements for use as the conductive element in pads for health monitoring and diagnostic applications and other wearable monitoring systems.

Applications of the disclosed electrodes and wiring elements include remote healthcare, including wearable monitoring devices; detection of adverse health events including heart attack, atrial fibrillation, stroke, and the like; sports performance; military applications; and the like. The electrodes or wiring elements can be incorporated into any wearable device such as a garment (e.g., wetsuit, compression shirt, etc.), footwear (e.g. sneakers, shoes, boots, etc.), headwear, or a smaller wearable device (wrist band, head band, chest strap, belt, etc.). The wearable monitoring device can be worn by the user to receive signals (bio-potentials, performance, etc.) that can be recorded and tracked by a fixed computer or a mobile device such as a smart watch, smart phone, etc. using wireless technology such as Bluetooth.

Advantages of using the conductive polymer fabric electrodes or wiring elements is that it can provide the monitoring device with the feel and breathability of fabric while at the same time functioning as a sufficient conductor comparable to metals. As the device will not be made from metal, it meets the requirements of various industries in the wearable electronics field.

The disclosed monitoring systems can be used to measure bio-potentials including ECG (for the measure of heart activity), electroencephalogram (EEG; for the measure of brain activity), and electromyography (EMG; for the measure of muscle activity) by measuring electric potentials on the surface of living tissue over time. Other types of monitoring, such as heart rate, respiratory rate, bioelectrical impedance analysis (BIA), skin conductance measurements, and the like further can be conducted. BIA measures the electrical impedance of body tissues, which can then be used to calculate an estimate of total body water and in turn an estimate of body fat. Skin conductance or alternatively, electrodermal activity (EDA), are measurements of the active and passive electrical properties of the skin.

For any sensor, including systems such as EMG, EEG, and ECG, signal-to-noise ratio is an important feature in detecting a response. The minimal signal to noise ratio to have a discernable signal is 3. The bio-potential wearable monitoring systems disclosed herein have a signal to noise ratio of about 3 to about 30 dB, specifically about 10 to about 25 dB, and yet more specifically about 12 to about 20 dB. An exemplified ECG monitoring system disclosed herein was found to have a signal to noise ratio of 15.4 dB. Further as it pertains to ECG monitoring system, it is most desirable to have a range of measuring an ECG from about 40 to about 180 bpm in that the 180 bpm would be sufficient for covering the age range of 15 years to 100 years for people undergoing high intensity training. ECG at ranges less than this, such as a heart rate ranging from 50 bpm to 120 bpm, can serve many other applications such as detecting dysrhythmia for people at rest or during light activity.

The disclosed electrodes or wiring elements can be used in a heart rate monitoring (HRM) system, ECG system, EEG system, EMG system, BIA system, EDA system, and the like.

Wiring element may be defined as an electrical conductor or a system of wires providing electric circuits for a device.

A “fabric” is a manufactured assembly of interlacing fibers, filaments, and/or yarns having (1) substantial surface (planar) area in relation to its thickness, and (2) adequate mechanical strength to give it a cohesive structure. Most fabrics are knitted or woven, but some are produced by non-woven processes such as braiding, felting, and twisting. Applied loosely, ‘fabric’ also includes laces, meshes, and nets.

In an embodiment, an electrode or wiring element for use in a bio-potential wearable monitoring system comprises a conductive element embedded in a composite material, wherein the conductive element is a conductive polymer fabric.

Suitable materials that can be used to prepare the conductive polymer fabric electrodes and conductive polymer fabric wiring elements include those materials described in U.S. Patent Publication Nos. 2015/0017421A1 to Sotzing and 2014/0011004A1 to Sotzing; U.S. patent application Ser. No. 15/135,895 filed Apr. 22, 2016, Ser. No. 15/135,898 filed Apr. 22, 2016, and Ser. No. 15/135,894 filed Apr. 22, 2016; and International Patent Publication No. WO2015/138298A1 to Sotzing et al., each of which is incorporated by reference herein.

U.S. Patent Publication No. 2015/0017421A1 to Sotzing discloses suitable materials that can be used to prepare the conductive polymer fabric electrodes or conductive polymer fabric wiring elements, the relevant portions of which are reproduced below. Such materials include conductive synthetic leather, electrically conductive polymeric fibrous substrate, and electrically conductive polymeric fiber, each comprising polymeric fiber which in turn comprises desiccant particles, wherein a portion of the desiccant particles are located at the surface of the polymeric fiber. Herein, the desiccant particles are used interchangeably with “nucleophile derivatized nanoparticles”. The substrates are made electrically conductive by disposing an electrically conductive polymer onto the polymeric fiber where the electrically conductive polymer is at least in partial contact with the desiccant particles. Not wishing to be bound by theory, but it is believed there is an interaction between the electrically conductive polymer and the desiccant particles which allows the coated substrate to achieve sheet resistances ranging from 0.4 to 400 Ohms/square. For example, it has been found that electrically conductive synthetic leather does not require expensive metals such as silver to obtain very low sheet resistances (1.5 Ohms/square). Commercial silver fabric is able to obtain 1 Ohm/square sheet resistance but it is costly, being prepared from a precious metal.

In an embodiment, an electrically conductive synthetic leather is prepared from poly(3,4-ethylenedioxythiophene)-polystyrenesulfonate (PEDOT-PSS).

In an embodiment, the fibrous substrate is a non-woven fibrous substrate. In an embodiment, the fibrous substrate is a synthetic leather or a synthetic suede. In another embodiment, the fibrous substrate is woven.

In an embodiment is an electrically conductive synthetic leather comprising a synthetic leather comprising polymeric fibers comprising desiccant particles wherein a portion of the desiccant particles are located at the surface of the polymeric fibers; and an electrically conductive polymer film disposed on at least a portion of the polymeric fibers and at least in partial contact with the desiccant particles.

In another embodiment, an electrically conductive fibrous substrate comprises a fibrous substrate comprising polymeric fibers comprising desiccant particles wherein a portion of the desiccant particles are located at the surface of the polymeric fiber; and an electrically conductive polymer film disposed on at least a portion of the polymeric fibers of the fibrous substrate and at least in partial contact with the desiccant particles.

In another embodiment, an electrically conductive fiber comprises a polymeric fiber comprising desiccant particles wherein a portion of the desiccant particles are located at the surface of the polymeric fiber; and an electrically conductive polymer film disposed on at least a portion of the polymeric fiber and at least in partial contact with the desiccant particles.

In an embodiment, the electrically conductive synthetic leather or the electrically conductive fibrous substrate exhibits semiconductive behavior at low temperature (e.g. below 0° C.) and metallic behavior at high temperature (e.g. above 0° C.).

In an embodiment, the electrically conductive synthetic leather or the electrically conductive fibrous substrate exhibits sheet resistances ranging from 0.4 to 400 Ohms/square.

Exemplary electrically conductive polymers that can be used to prepare the electrically conductive synthetic leather, electrically conductive fibrous substrate, and electrically conductive fiber include poly(3,4-ethylenedioxythiophene) (“PEDOT”) including poly(3,4-ethylenedioxythiophene):poly(styrenesulfonate) (“PEDOT:PSS”) aqueous dispersion, a substituted poly(3,4-ethylenedioxythiophene), a poly(thiophene), a substituted poly(thiophene), a poly(pyrrole), a substituted poly(pyrrole), a poly(aniline), a substituted poly(aniline), a poly(acetylene), a poly(p-phenylenevinylene) (PPV), a poly(indole), a substituted poly(indole), a poly(carbazole), a substituted poly(carbazole), a poly(azepine), a (poly)thieno[3,4-b]thiophene, a substituted poly(thieno[3,4-b]thiophene), a poly(dithieno[3,4-b:3′,4′-d]thiophene), a poly(thieno[3,4-b]furan), a substituted poly(thieno[3,4-b]furan), a derivative thereof, a combination thereof, and the like.

The electrically conductive polymer can be used in an amount of about 0.1 to about 10.0 wt % based on the weight of the substrate, specifically about 0.2 to about 8.0 wt %, more specifically about 0.3 to about 7.0 wt % and yet more specifically about 0.5 to about 5.0 wt %. In the fiber embodiment, the electrically conductive polymer can be used in an amount of about 0.1 to about 10.0 wt % based on the weight of the fiber, specifically about 0.2 to about 8.0 wt %, more specifically about 0.3 to about 7.0 wt % and yet more specifically about 0.5 to about 5.0 wt %.

The electrically conductive polymer film coating on the electrically conductive synthetic leather or the electrically conductive fibrous substrate can have an average thickness of 300 nm or less, specifically 250 nm or less, more specifically 100 nm or less, yet more specifically 30 nm or less, still yet more specifically 25 nm or less, and even more specifically 20 nm or less. The lower end of the thickness range can be about 4 nm or more.

Artificial leather can be made from any polymeric material such as nylon 6, nylon 66, nylon 610, nylon 12, co-polymerized nylon, polyethylene terephthalate, polytrimethylene terephthalate, spandex (polyurethane-polyurea copolymer), polybutylene terephthalate, polypropylene terephthalate, polyurethane, polypropylene, polyethylene, polyester-based polyurethane copolymers thereof, or a combination thereof. The artificial leather can be finished (material having a glossy surface) or unfinished (material without a glossy surface). In an embodiment, a desiccant is used to prepare the artificial leather such that the fibers of the artificial leather comprise desiccant particles wherein a portion of the desiccant particles are located at the surface of the synthetic leather fibers.

The polymeric fiber and fibrous substrate can be made from any polymeric material such as nylon 6, nylon 66, nylon 610, nylon 12, co-polymerized nylon, polyethylene terephthalate, polytrimethylene terephthalate, spandex (polyurethane-polyurea copolymer), polybutylene terephthalate, polypropylene terephthalate, polyurethane, polypropylene, polyethylene, polyester-based polyurethane, copolymers thereof, or a combination thereof. In an embodiment, a desiccant is used to prepare the fibrous substrate such that the fibers comprise desiccant particles wherein a portion of the desiccant particles are located at the surface of the fiber.

Exemplary desiccants include inorganic oxides such as silica/silicon dioxide (SiO2), titania/titanium dioxide (TiO2), alumina/aluminum oxide, calcium oxide, or a combination thereof. In a further embodiment, the desiccant is in particulate form having average particle size of about 1 nanometer (nm) to about 5 micrometer, specifically about 10 nm to about 500 nm, and more specifically about 25 nm to about 200 nm.

The desiccant/nucleophile derivatized nanoparticles can be present on the surface of the polymeric fiber in an amount of about 0.01 to about 6.0% area relative to the total surface area of the polymeric fiber comprising desiccant/nucleophile derivatized nanoparticles, specifically about 0.05 to about 5.0% area, and more specifically about 0.1 to about 4.0%.

The electrically conductive substrate, including electrically conductive synthetic leather, is easily scalable to high volume manufacture. The electrically conductive polymer can be applied to the synthetic leather, fibrous substrate, or fiber using a variety of different techniques. For example drop casting, spray coating, ink jet printing, dip coating, gravure coating methods, and extrusion coating. Another approach is a soaking process. Many of these processes are easily adaptable to large scale manufacture.

These coating techniques generally comprise forming a mixture of the material to be coated with a solvent, applying the mixture to a surface of the synthetic leather substrate, and removing the solvent to form a thin film of the material adheringly disposed on the surface of the synthetic leather substrate. The solvent can be water, an organic solvent, or a combination of an organic solvent and water. Exemplary organic solvents include dimethyl sulfoxide (DMSO), dichloromethane (DCM), toluene, N,N-dimethyl formamide (DMF), propylene glycol monomethyl ether acetate (PGMEA), propylene glycol monomethyl ether (PGME), acetone, methanol, and ethanol.

The mixture can contain the electrically conductive polymer at a concentration of about 0.1 weight percent (wt. %) to about 5 wt. %, based on the total weight of the mixture, specifically about 0.2 to about 4 wt. %, more specifically 0.3 to about 4 wt. %, and still more specifically about 1.0 to about 3 wt. %.

In an embodiment, the artificial leather, polymeric fiber, and fibrous substrate can be plasma treated prior to the application of the electrically conductive polymer. Plasma or other surface modification treatment can be used to impart good wettability and adhesion of the electrically conductive polymer on the surface of artificial leather, polymeric fiber, and fibrous substrate. In an exemplary plasma treatment process, the use of atmospheric pressure plasma (helium, argon, air, oxygen, or a combination thereof) can be used. Other exemplary surface modification includes exposing the artificial leather, polymeric fiber, and fibrous substrate to organic solvents with similar solubility parameters as DMSO. Solvent treatment can be conducted alone or in addition to plasma treatment.

A method of making an electrically conductive synthetic leather, an electrically conductive fibrous substrate, or an electrically conductive fiber comprising disposing an electrically conductive polymer onto a synthetic leather comprising polymeric fibers comprising desiccant particles wherein a portion of the desiccant particles are located at the surface of the polymeric fibers, onto a fibrous substrate comprising polymeric fibers comprising desiccant particles wherein a portion of the desiccant particles are located at the surface of the polymeric fiber, or onto a polymeric fiber comprising desiccant particles wherein a portion of the desiccant particles are located at the surface of the polymeric fiber. In an embodiment, prior to the disposing step, the synthetic leather, the fibrous substrate, and the polymeric fiber are surface treated with a plasma treatment, a solvent treatment, or a combination thereof.

In an embodiment, PEDOT:PSS aqueous dispersion is loaded into unfinished and finished polyethylene terephthalate leather to yield highly conductive leather samples having sheet resistances ranging from 0.4 to 400 Ohms/square.

The term “fiber” as used herein includes single filament and multi-filament fibers, i.e., fibers spun, woven, knitted, crocheted, knotted, pressed, plied, or the like from multiple filaments. No particular restriction is placed on the length of the fiber, other than practical considerations based on manufacturing considerations and intended use. Similarly, no particular restriction is placed on the width (cross-sectional diameter) of the fibers, other than those based on manufacturing and use considerations. The width of the fiber can be essentially constant, or vary along its length. For many purposes, the fibers can have a largest cross-sectional diameter of 2 nanometers and larger, for example up to 2 centimeters, specifically from 5 nanometers to 1 centimeter. In an embodiment, the fibers can have a largest cross-sectional diameter of 5 to 500 micrometers, more particularly, 5 to 200 micrometers, 5 to 100 micrometers, 10 to 100 micrometers, 20 to 80 micrometers, or 40 to 50 micrometers. In one embodiment, the conductive fiber has a largest circular diameter of 40 to 45 micrometers. Further, no restriction is placed on the cross-sectional shape of the fiber. For example, the fiber can have a cross-sectional shape of a circle, ellipse, square, rectangle, or irregular shape.

U.S. patent application Ser. No. 15/135,898 filed Apr. 22, 2016 discloses suitable materials that can be used to prepare the conductive polymer fabric electrodes or conductive polymer fabric wiring elements, the relevant portions of which are reproduced below. U.S. patent application Ser. No. 15/135,898 discloses stretchable organic metals, more specifically organic stretchable electrically conductive structures exhibiting metallic properties. The stretchable electrically conductive structures comprise a stretchable insulating substrate comprising nucleophile derivatized nanoparticles located at the surface of the stretchable insulating substrate and a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate. The stretchable insulating substrate can be a stretchable fiber or stretchable fabric. It has been surprisingly found that the conducting polymer:template polymer coating of the stretchable electrically conductive structure has the ability to stretch along with the stretchable insulating substrate, whereas the conducting polymer alone will only tear. The stretchable electrically conductive structure has the ability to be stretched and retain its metal-like properties with respect to its temperature characteristics, ohmic behavior, and high charge carrier mobilities and concentrations.

In these structures, the coating of conducting polymer:template polymer material is phase separated such that there is a higher concentration of template polymer at the interface of the surface of the stretchable insulating substrate and the coating of conducting polymer:template polymer material. Not wishing to be bound by theory, it is proposed that a small amount of ‘leaving group’ or ‘nucleophile’ derivatized nanoparticle present at the surface of the stretchable insulating substrate reacts with the template polymer, e.g. polystyrenesulfonic acid, polyvinylphoshoric acid, etc. used as a counterion for the conducting polymer, to form covalent bonds at the particle surface that induce a phase segregation of the template polymer from the conducting polymer. This phase segregation generates a gradient of ratios of Template Polymer:Conducting Polymer, with the highest amount of Template Polymer:Conducting polymer occurring at the interface of the substrate and the Template Polymer:Conducting polymer film. Thus, there is a gradient by which most of the conducting polymer is at the surface of the film of conducting polymer:template polymer material, and away from the substrate surface. This nanoparticle induced phase separation, which leads to low sheet resistances, can be used to make organic wires and biopotential electrodes, having current carrying capacities approaching copper.

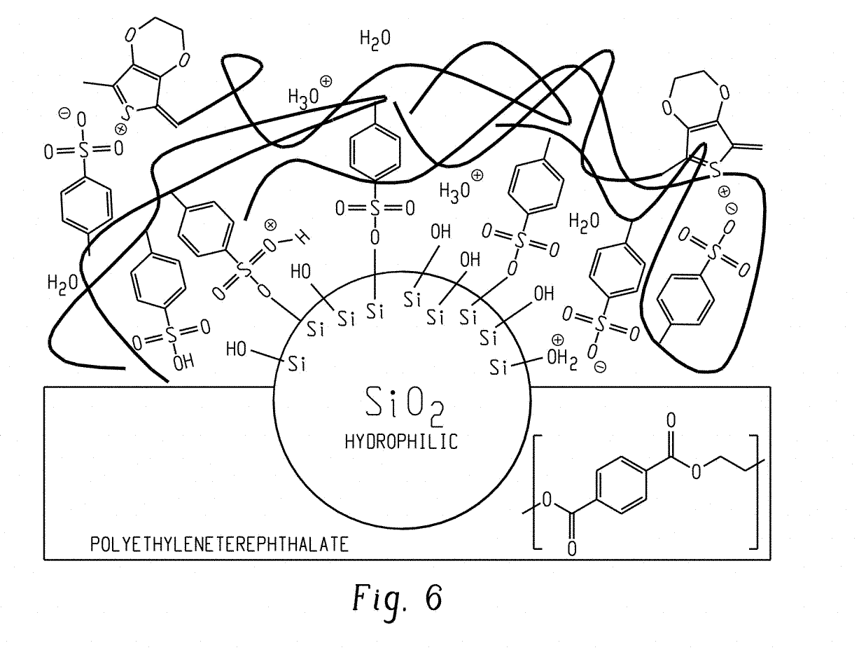

FIG. 6 depicts the proposed phase segregation of PEDOT:PSS and is not to scale. The substrate is polyethyleneterephthalate (PET) comprising particles of silica at the surface (a single SiO₂ nanoparticle is shown). The PSS reacts with the Si—OH groups of the silica to form covalent bonds, thus resulting in a higher concentration of PSS at the interface of the PET substrate and PEDOT:PSS film and a higher concentration of PEDOT furthest from this interface. Not wishing to be bound by theory, but it is possible that the phase segregation occurs as the nucleophile derivatized nanoparticle is in a solid phase and the template polymer is in solution; and it is surprising that such a chemical reaction would take place and induce a phase separation phenomenon.

The stretchable electrically conductive structure exhibits certain mechanical properties, e.g. elasticity, as well as a certain ionic conductivity, even upon stretching and repeated stretching. The stretchable electrically conductive structure is both flexible and expandable owing to the stretchable insulating substrate.

Stretchability for a given material can be characterized by elongation at break and the ability of elastic recovery. Elastomeric material such as Spandex, have a large elongation at break value (up to about 800% to about 900%) and recover to their original form when the force is removed within a certain range. Different fabrics have different stretchability depending on the type, fiber/yarn diameter, fiber bundle, etc. In general, common fabrics, such as silk or cotton, have little to no stretchability as compared to Spandex. However, there are many commercially available products which contain small amount of Spandex (about 5-15%) that have sufficient stretchability for use as stretchable insulating substrates herein.

It has further surprisingly been found that the stretchable electrically conductive structure comprising the conducting polymer:template polymer coating is washable. “Washable” means that the stretchable electrically conductive structure has the ability to maintain functionality and not be damaged after being soaking in water or other suitable solvent with or without laundry detergent/soap and/or agitation, followed by an optional rinsing step and subsequent drying or removal of the solvent. In one embodiment, the washable stretchable electrically conductive structure further comprises a hydrophobic fabric treatment, for example Scotchgard™ Fabric & Upholstery Protector, perfluorinated alkylsulfonate (e.g. wherein the “alkyl” is a C4-C9), perfluorinated urethanes, and the like.

The stretchable insulating substrate can be a stretchable fiber or stretchable fabric comprising nucleophile derivatized nanoparticles located at least at the surface of the stretchable insulating substrate.

The stretchable fabric can be woven or non-woven fabric comprising fibers of stretchable polymeric material.

The stretchable insulating substrate can be a stretchable fiber or stretchable fabric. The stretchable insulating substrate comprises nucleophile derivatized nanoparticles at the substrate surface. The term “substrate comprising nucleophile derivatized nanoparticles at the substrate surface” is synonymous with the term “substrate comprising surface nucleophile derivatized nanoparticles”. Further as used herein, the term “surface nucleophile derivatized nanoparticles” is synonymous with “surface nanoparticles”.

Suitable stretchable insulating substrate materials include stretchable polymeric material. Exemplary stretchable polymeric material include polyurethane, polyester-polyurethane copolymer (e.g. Spandex), and blends of polyurethane or polyester-polyurethane and an additional synthetic organic polymer e.g., polyacrylic, polyamide (nylon), polycarbonate, polyether, polyester (e.g. polyethyleneterephthalate), polyethylene, polyimide, polyurethane, polyester-polyurethane copolymer, polyurea, polythiourea, polysiloxane, polyisoprene, polybutadiene, polyethylene oxide, polylactic acid blends thereof, copolymers thereof and the like. In another embodiment, fabrics prepared from a combination of stretchable fibers (e.g., polyester-polyurethane (Spandex)) fibers and other fibers (e.g. synthetic organic polymers or natural materials (e.g., cotton, silk, and wool)) can be used as long as the overall fabric is stretchable.

The nucleophile derivatized nanoparticles can be nanoparticulate inorganic oxide such as silica/silicon dioxide (SiO₂), titania/titanium dioxide (TiO₂), alumina/aluminum oxide, calcium oxide, amine functionalized nanoparticles, or a combination thereof. The nucleophile derivatized nanoparticles can have an average particle size of about 1 nanometer (nm) to about 1000 nm, specifically about 5 nm to about 500 nm, and more specifically about 10 nm to about 200 nm. In an embodiment, the nucleophile derivatized nanoparticles have an average particle size measured by transmission electron microscopy of about 10 nm, with a distribution of about 8 to about 12 nm.

The nucleophile derivatized nanoparticles can be present in an amount of about 0.01 to about 6.0 wt % by weight of the stretchable insulating substrate comprising nucleophile derivatized nanoparticles, specifically about 0.05 to about 5.0 wt %, and more specifically about 0.1 to about 4.0 wt % by weight of the stretchable insulating substrate comprising nucleophile derivatized nanoparticles.

The nucleophile derivatized nanoparticles can be present on the surface of the stretchable insulating substrate in an amount of about 0.01 to about 6.0% area relative to the total surface area of the stretchable insulating substrate comprising nucleophile derivatized nanoparticles, specifically about 0.05 to about 5.0% area, and more specifically about 0.1 to about 4.0% area relative to the total surface area of the stretchable insulating substrate comprising nucleophile derivatized nanoparticles.

The stretchable insulating substrate comprising surface nucleophile derivatized nanoparticles can be of any thickness. For those applications that require flexibility and/or stretchability, the thickness of the stretchable insulating substrate comprising surface nucleophile derivatized nanoparticles can be about 100 nm to about 1 centimeter (cm), specifically about 500 nm to about 0.1 cm, more specifically about 1 micrometer to about 5 millimeter (mm). In an embodiment, a stretchable insulating substrate can have a thickness of about 1 micrometer to about 5 mm.

The nucleophile derivatized nanoparticles can be present at the substrate surface in a random pattern or an organize pattern or design. The nucleophile derivatized nanoparticles are present at least embedded at the surface of the substrate where at least a portion of the nanoparticle is exposed, and optionally further distributed within the substrate material itself.

Nucleophile derivatized nanoparticles are incorporated into the stretchable insulating substrate such that the nanoparticles are exposed to the surface. Treatment, such as plasma treatment, can further expose the nanoparticles as well as generate a more polar polymer surface. Plasma treatment can be conducted using processes and process conditions well known in the art. The nucleophile derivatized nanoparticle serves as nucleation sites and allow growth or have segregation better achieved by polarity induced on polymer due to plasma treatment.

To form the substrate comprising surface nucleophile derivatized nanoparticles, the nanoparticles can be incorporated into a substrate material any number of ways. In one embodiment, the substrate material is combined with nanoparticles at or slightly above the melt temperature of the substrate material and blended with high shear to ensure no clustering of the nanoparticles. The resulting melt can be processed via conventional melt processing, melt spinning, and/or extrusion techniques known in the art.

In another embodiment, the nanoparticles can be applied to a substrate via a deposition technique. For example, silica nanoparticles having exposed hydroxyl functionality could be ‘blown’ onto a PET/Spandex substrate, and then the nanoparticles could undergo a transesterification with the PET that would covalently link the silica particles and anchor them to the PET/Spandex substrate surface.

The conducting polymer film structure comprises a conducting polymer:template polymer film disposed on at least a portion of the surface of the stretchable insulating substrate comprising surface nucleophile derivatized nanoparticles. In an alternative embodiment, the conducting polymer:template polymer coating is in the form of a pattern on at least a portion of the surface of the stretchable insulating substrate.

A conducting polymer:template polymer coating can be formed on the stretchable insulating substrate comprising surface nanoparticles using any variety of techniques known in the art. For example, a PEDOT-PSS film can be formed by using solution processing techniques. The stretchable insulating substrate can be soaked with a dispersion of PEDOT-PSS in a suitable solvent followed by drying and/or annealing. Other suitable processes include drop casting, tape casting, flow coating, spray coating, etc. The annealing can be conducted at temperatures of about 80 to about 130° C., specifically about 90 to about 125° C., and yet more specifically about 100 to about 120° C. for as long as needed. Such conditions can be carried out in an oven or other suitable apparatus with or without vacuum or air flow.

Conducting polymers are known in the art and are often complexed with a template polymer, e.g. polystyrenesulfonic acid, polyvinylphoshoric acid, etc. where the sulfate or phosphonate, etc. serve as the counterion for the conducting polymer that possess positive charges as the charge carrier.

Conducting polymers include those conducting polymers comprising units of conducting monomers, e.g. where the conducting polymer is prepared by template polymerization. Examples of suitable conducting monomers include those known in the art to exhibit electroactivity when polymerized, including but not limited to thiophene, substituted thiophene, 3,4-ethylenedioxythiophene, thieno[3,4-b]thiophene, substituted thieno[3,4-b]thiophene, dithieno[3,4-b:3′,4′-d]thiophene, thieno[3,4-b]furan, substituted thieno[3,4-b]furan, bithiophene, substituted bithiophene, pyrrole, substituted pyrrole, phenylene, substituted phenylene, naphthalene, substituted naphthalene, biphenyl and terphenyl and their substituted versions, phenylene vinylene, substituted phenylene vinylene, aniline, substituted aniline, the monomers disclosed herein as structures (I)-(XXIX), combinations thereof, and the like.

Suitable conducting monomers include 3,4-ethylenedioxythiophene, 3,4-ethylenedithiathiophene, 3,4-ethylenedioxypyrrole, 3,4-ethylenedithiapyrrole, 3,4-ethylenedioxyfuran, 3,4-ethylenedithiafuran, and derivatives having the general structure (I):

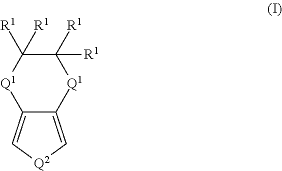

wherein each occurrence of Q¹ is independently S or O; Q² is S, O, or N—R² wherein R² is hydrogen or C₁-C₆ alkyl; and each occurrence of R¹ is independently hydrogen, C₁-C₁₂ alkyl, C₁-C₁₂ alkyl-OH, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl. In one embodiment, each occurrence of R¹ is hydrogen. In one embodiment, each Q¹ is O and Q² is S. In another embodiment, each Q¹ is O, Q² is S, and one R¹ is C₁-C₁₂ alkyl, C₁-C₁₂ alkyl-OH, C₁-C₁₂ haloalkyl, C₁-C₁₂ alkoxy, C₁-C₁₂ haloalkoxy, C₁-C₆ alkyl-O—C₁-C₆ alkyl, while the remaining R¹ are hydrogen. In another embodiment, each Q¹ is O, Q² is S, and one R¹ is C₁ alkyl-OH, while the remaining R¹ are hydrogen. A specific conducting monomer is EDOT.

Another suitable conducting monomer includes an unsubstituted and 2- or 6-substituted thieno[3,4-b]thiophene and thieno[3,4-b]furan having the general structures (II), (III), and (IV):

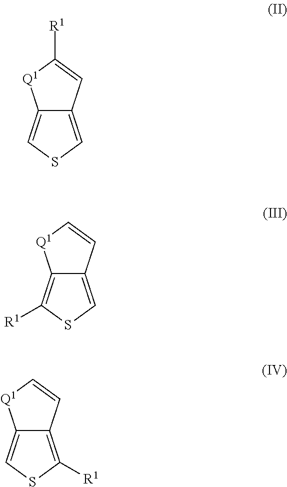

wherein Q¹ is S, O, or Se; and R¹ is hydrogen, C₁-C₁₂ alkyl, C₁-C₁₂ haloalkyl including perfluoroalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl. In one embodiment, Q¹ is S and R¹ is hydrogen. In another embodiment, Q¹ is O and R¹ is hydrogen. In yet another embodiment, Q¹ is Se and R¹ is hydrogen.

Another suitable conducting monomer includes substituted 3,4-propylenedioxythiophene (ProDOT) monomers according to the general structure (V):

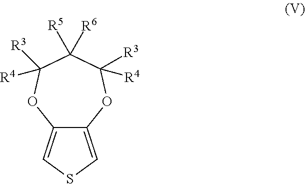

wherein each instance of R³, R⁴, R⁵, and R⁶ independently is hydrogen; optionally substituted C₁-C₂₀ alkyl, C₁-C₂₀ haloalkyl, aryl, C₁-C₂₀ alkoxy, C₁-C₂₀ haloalkoxy, aryloxy, C₁-C₁₀ alkyl-O—C₁-C₁₀ alkyl, —C₁-C₁₀ alkyl-O-aryl, —C₁-C₁₀ alkyl-aryl; or hydroxyl. The C₁-C₂₀ alkyl, C₁-C₂₀ haloalkyl, aryl, C₁-C₂₀ alkoxy, C₁-C₂₀ haloalkoxy, aryloxy, —C₁-C₁₀ alkyl-O—C₁-C₁₀ alkyl, —C₁-C₁₀ alkyl-O-aryl, or —C₁-C₁₀ alkyl-aryl groups each may be optionally substituted with one or more of C₁-C₂₀ alkyl; aryl; halogen; hydroxyl; —N—(R²)₂ wherein each R² is independently hydrogen or C₁-C₆ alkyl; cyano; nitro; —COOH; —S(═O)C₀-C₁₀ alkyl; or —S(═O)₂C₀-C₁₀ alkyl. In one embodiment, R⁵ and R⁶ are both hydrogen. In another embodiment, R⁵ and R⁶ are both hydrogen, each instance of R³ independently is C₁-C₁₀ alkyl or benzyl, and each instance of R⁴ independently is hydrogen, C₁-C₁₀ alkyl, or benzyl. In another embodiment, R⁵ and R⁶ are both hydrogen, each instance of R³ independently is C₁-C₅ alkyl or benzyl and each instance of R⁴ independently is hydrogen, C₁-C₅ alkyl, or benzyl. In yet another embodiment, each instance of R³ and R⁴ are hydrogen, and one of R⁵ and R⁶ is hydroxyl while the other is hydrogen.

Other suitable conducting monomers include pyrrole, furan, thiophene, and derivatives having the general structure (VI):

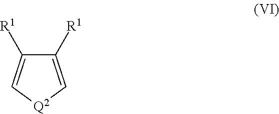

wherein Q² is S, O, or N—R² wherein R² is hydrogen or C₁-C₆ alkyl; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Additional conducting monomers include isathianaphthene, pyridothiophene, pyrizinothiophene, and derivatives having the general structure (VII):

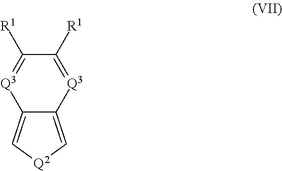

wherein Q² is S, O, or N—R² wherein R² is hydrogen or C₁-C₆ alkyl; each occurrence of Q³ is independently CH or N; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Still other conducting monomers include oxazole, thiazole, and derivatives having the general structure (VIII):

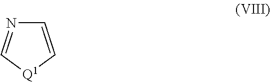

wherein Q¹ is S or O.

Additional conducting monomers include the class of compounds according to structure (IX):

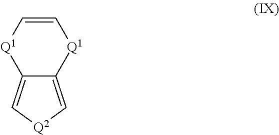

wherein Q² is S, O, or N—R² wherein R² is hydrogen or C₁-C₆ alkyl; and each occurrence of Q¹ is independently S or O.

Additional conducting monomers include bithiophene, bifuran, bipyrrole, and derivatives having the following general structure (X):

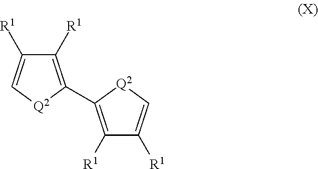

wherein each occurrence of Q² is independently S, O, or N—R² wherein R² is hydrogen or C₁-C₆ alkyl; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Conducting monomers include terthiophene, terfuran, terpyrrolo, and derivatives having the following general structure (XI):

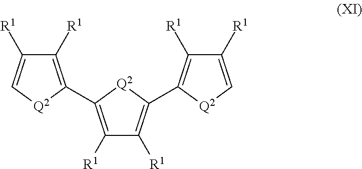

wherein each occurrence of Q² is independently S, O, or N—R² wherein R² is hydrogen or C₁-C₆ alkyl; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Additional conducting monomers include thienothiophene, thienofuran, thienopyrrole, furanylpyrrole, furanylfuran, pyrolylpyrrole, and derivatives having the following general structure (XII):

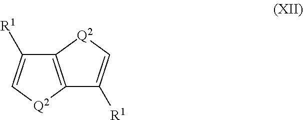

wherein each occurrence of Q² is independently S, O, or N—R² wherein R² is hydrogen or C₁-C₆ alkyl; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Still other conducting monomers include dithienothiophene, difuranylthiophene, dipyrrolylthiophene, dithienofuran, dipyrrolylfuran, dipyrrolylpyrrole, and derivatives having the following general structure (XIII):

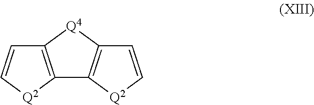

wherein each occurrence of Q² is independently S, O, or N—R² wherein R² is hydrogen or C₁-C₆ alkyl; Q⁴ is C(R¹)₂, S, O, or N—R²; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Additional conducting monomers include dithienylcyclopentenone, difuranylcyclopentenone, dipyrrolylcyclopentenone and derivatives having the following general structure (XIV):

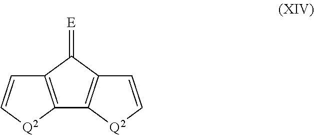

wherein each occurrence of Q² is independently S, O, or N—R² wherein R² is hydrogen or C₁-C₆ alkyl; and E is O or C(R⁷)₂, wherein each occurrence of R⁷ is an electron withdrawing group.

Other suitable conducting monomers include those having the following general structure (XV):

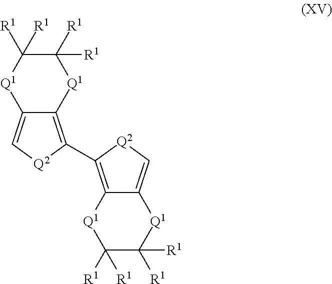

wherein each occurrence of Q¹ is independently S or O; each occurrence of Q² is independently S, O, or N—R² wherein R² is hydrogen or C₁-C₆ alkyl; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl. In one embodiment, each occurrence of Q¹ is O; each occurrence of Q² is S; and each occurrence of R¹ is hydrogen.

Additional conducting monomers include dithienovinylene, difuranylvinylene, and dipyrrolylvinylene according to the structure (XVI):

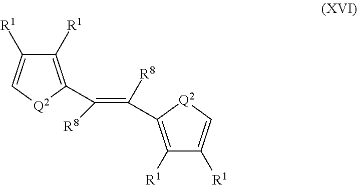

wherein each occurrence of Q² is independently S, O, or N—R² wherein R² is hydrogen or C₁-C₆ alkyl; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl; and each occurrence of R⁸ is hydrogen, C₁-C₆ alkyl, or cyano.

Other conducting monomers include 1,2-trans(3,4-ethylenedioxythienyl)vinylene, 1,2-trans(3,4-ethylenedioxyfuranyl)vinylene, 1,2-trans(3,4ethylenedioxypyrrolyl)vinylene, and derivatives according to the structure (XVII):

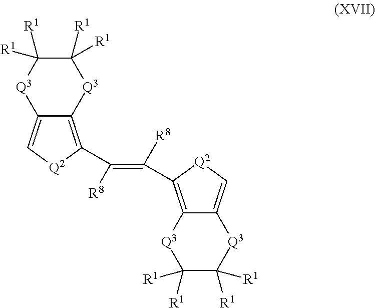

wherein each occurrence of Q³ is independently CH₂, S, or O; each occurrence of Q² is independently S, O, or N—R² wherein R² is hydrogen or C₁-C₆ alkyl; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl; and each occurrence of R⁸ is hydrogen, C₁-C₆ alkyl, or cyano.

Additional conducting monomers include the class bis-thienylarylenes, bis-furanylarylenes, bis-pyrrolylarylenes and derivatives according to the structure (XVIII):

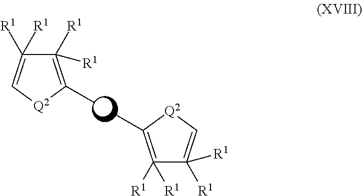

wherein each occurrence of Q² is independently S, O, or N—R² wherein R² is hydrogen or C₁-C₆ alkyl; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl; and

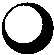

represents an aryl. Exemplary aryl groups include furan, pyrrole, N-substituted pyrrole, phenyl, biphenyl, thiophene, fluorene, 9-alkyl-9H-carbazole, and the like.

Other conducting monomers include the class of bis(3,4 ethylenedioxythienyl)arylenes, related compounds, and derivatives according to the structure (XIX):

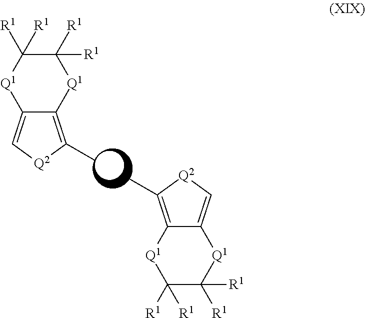

wherein each occurrence of Q¹ is independently S or O; each occurrence of Q² is independently S, O, or N—R² wherein R² is hydrogen or C₁-C₆ alkyl; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl; and

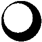

represents an aryl.

Other exemplary conducting monomers include bis(3,4 ethylenedioxythienyl)arylenes according to structure (XIX) includes the compound wherein all Q1 are O, both Q2 are S, all R1 are hydrogen, and

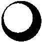

is phenyl linked at the 1 and 4 positions.

Another exemplary compound is where all Q1 are O, both Q2 are S, all R1 are hydrogen, and

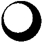

is thiophene linked at the 2 and 5 positions.

Additional conducting monomers include the class of compounds according to structure (XX):

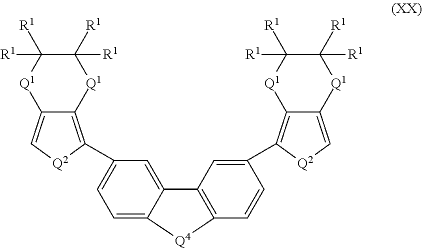

wherein each occurrence of Q¹ is independently S or O; each occurrence of Q² is independently S, O, or N—R² wherein R² is hydrogen or C₁-C₆ alkyl; Q⁴ is C(R¹)₂, S, O, or N—R²; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl. In one embodiment, each occurrence of Q¹ is O; each occurrence of Q² is S; each occurrence of R¹ is hydrogen; and R² is methyl.

Still other conducting monomers include the class of compounds according to structure (XXI):

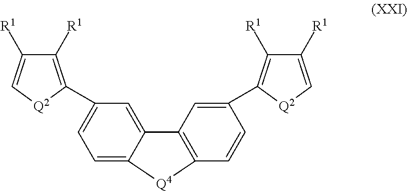

wherein each occurrence of Q² is independently S, O, or N—R² wherein R² is hydrogen or C₁-C₆ alkyl; Q⁴ is C(R′)₂, S, O, or N—R²; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Additional conducting monomers include the class of compounds according to structure (XXII):

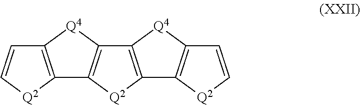

wherein each occurrence of Q² is independently S, O, or N—R² wherein R² is hydrogen or C₁-C₆ alkyl; each occurrence of Q⁴ is C(R′)₂, S, O, or N—R²; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Other exemplary monomers include the class of compounds according to structure (XXIII):

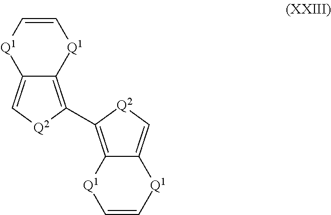

wherein each occurrence of Q² is independently S, O, or N—R² wherein R² is hydrogen or C₁-C₆ alkyl; and each occurrence of Q¹ is independently S or O.

Exemplary conducting monomers include the class of compounds according to structure (XXIV):

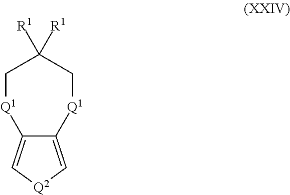

wherein Q² is S, O, or N—R² wherein R² is hydrogen or C₁-C₆ alkyl; each occurrence of Q¹ is independently S or O; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, —C₁-C₆ alkyl-aryl, —C₁-C₆ alkyl-O-aryl, or —C₁-C₆ alkyl-O-aryl. In one embodiment, one R¹ is methyl and the other R¹ is benzyl, —C₁-C₆ alkyl-O-phenyl, —C₁-C₆ alkyl-O-biphenyl, or —C₁-C₆ alkyl-biphenyl.

Additional conducting monomers include the class of compounds according to structure (XXV):

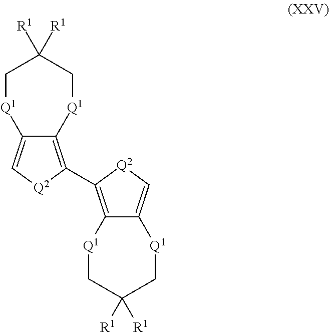

wherein each occurrence of Q² is independently S, O, or N—R² wherein R² is hydrogen or C₁-C₆ alkyl; each occurrence of Q¹ is independently S or O; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl. In one embodiment, one R¹ is methyl and the other R¹ is —C₁-C₆ alkyl-O-phenyl or —C₁-C₆ alkyl-O-biphenyl per geminal carbon center.

Other conducting monomers include the class of compounds according to structure (XXVI):

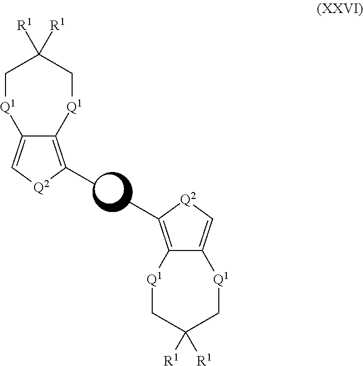

wherein each occurrence of Q² is independently S, O, or N—R² wherein R² is hydrogen or C₁-C₆ alkyl; each occurrence of Q¹ is independently S or O; each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl; and

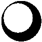

represents an aryl. In one embodiment, one R¹ is methyl and the other R¹ is —C₁-C₆ alkyl-O-phenyl or —C₁-C₆ alkyl-O-biphenyl per geminal carbon center.

Exemplary conducting monomers include the class of compounds according to structure (XXVII):

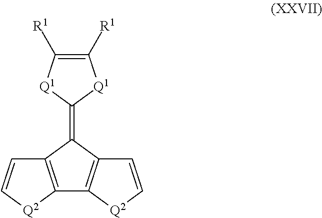

wherein each occurrence of Q² is independently S, O, or N—R² wherein R² is hydrogen or C₁-C₆ alkyl; each occurrence of Q¹ is independently S or O; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Additional conducting monomers include the class of compounds according to structure (XXVIII):

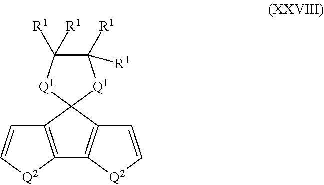

wherein each occurrence of Q² is independently S, O, or N—R² wherein R² is hydrogen or C₁-C₆ alkyl; each occurrence of Q¹ is independently S or O; and each occurrence of R¹ is independently hydrogen, C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, or —C₁-C₆ alkyl-O-aryl.

Another conducting monomer includes aniline or substituted aniline according to structure (XXIX):

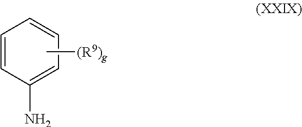

wherein g is 0, 1, 2, or 3; and each occurrence of R⁹ is independently C₁-C₁₂ alkyl, C₁-C₁₂ haloalkyl, C₁-C₁₂ alkoxy, C₁-C₁₂ haloalkoxy, aryl, —C₁-C₆ alkyl-O—C₁-C₆ alkyl, —C₁-C₆ alkyl-O-aryl, or N—R² wherein R² is hydrogen or C₁-C₆ alkyl.

The number average molecular weight (Me) of the conducting polymer can be in the range from about 1,000 to about 40,000, specifically from about 2000 to about 30,000.

The template polymerization may be conducted using a single type of conducting monomer to form a homopolymer, or two or more conducting monomer types in a copolymerization process to form a conducting copolymer. As used herein “conducting polymer” is inclusive of conducting homopolymers and conducting copolymers unless otherwise indicated. Furthermore, in one embodiment, the template polymerization may be conducted with a mixture of conducting monomers and nonconducting monomers as long as the resulting copolymer is conductive.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, “—CHO” is attached through carbon of the carbonyl group.

Unless otherwise indicated, the term “substituted” as used herein means replacement of one or more hydrogens with one or more substituents. Suitable substituents include, for example, hydroxyl, C₆-C₁₂ aryl, C₃-C₂₀ cycloalkyl, C₁-C₂₀ alkyl, halogen, C₁-C₂₀ alkoxy, C₁-C₂₀ alkylthio, C₁-C₂₀ haloalkyl, C₆-C₁₂ haloaryl, pyridyl, cyano, thiocyanato, nitro, amino, C₁-C₁₂ alkylamino, C₁-C₁₂ aminoalkyl, acyl, sulfoxyl, sulfonyl, amido, or carbamoyl.

As used herein, “alkyl” includes straight chain, branched, and cyclic saturated aliphatic hydrocarbon groups, having the specified number of carbon atoms, generally from 1 to about 20 carbon atoms, greater than 3 for the cyclic. Alkyl groups described herein typically have from 1 to about 20, specifically 3 to about 18, and more specifically about 6 to about 12 carbons atoms. Examples of alkyl include, but are not limited to, methyl, ethyl, n-propyl, isopropyl, n-butyl, 3-methylbutyl, t-butyl, n-pentyl, and sec-pentyl. As used herein, “cycloalkyl” indicates a monocyclic or multicyclic saturated or unsaturated hydrocarbon ring group, having the specified number of carbon atoms, usually from 3 to about 10 ring carbon atoms. Monocyclic cycloalkyl groups typically have from 3 to about 8 carbon ring atoms or from 3 to about 7 carbon ring atoms. Multicyclic cycloalkyl groups may have 2 or 3 fused cycloalkyl rings or contain bridged or caged cycloalkyl groups. Examples of cycloalkyl groups include cyclopropyl, cyclobutyl, cyclopentyl, or cyclohexyl as well as bridged or caged saturated ring groups such as norbornane or adamantane.

As used herein “haloalkyl” indicates both branched and straight-chain alkyl groups having the specified number of carbon atoms, substituted with 1 or more halogen atoms, generally up to the maximum allowable number of halogen atoms (“perhalogenated”). Examples of haloalkyl include, but are not limited to, trifluoromethyl, difluoromethyl, 2-fluoroethyl, and penta-fluoroethyl.

As used herein, “alkoxy” includes an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Haloalkoxy” indicates a haloalkyl group as defined above attached through an oxygen bridge.

As used herein, the term “aryl” indicates aromatic groups containing only carbon in the aromatic ring or rings. Such aromatic groups may be further substituted with carbon or non-carbon atoms or groups. Typical aryl groups contain 1 or 2 separate, fused, or pendant rings and from 6 to about 12 ring atoms, without heteroatoms as ring members. Where indicated aryl groups may be substituted. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl.

As used herein “heteroaryl” indicates aromatic groups containing carbon and one or more heteroatoms chosen from N, O, and S. Exemplary heteroaryls include oxazole, pyridine, pyrazole, thiophene, furan, isoquinoline, and the like. The heteroaryl groups may be substituted with one or more substituents.

As used herein, “halo” or “halogen” refers to fluoro, chloro, bromo, or iodo.

As used herein, “arylene” includes any divalent aromatic hydrocarbon or two or more aromatic hydrocarbons linked by a bond, a heteroatom (e.g., O, S, S(═O), S(═O)2, etc.), a carbonyl group, an optionally substituted carbon chain, a carbon chain interrupted by a heteroatom, and the like.

Poly(3,4-ethylenedioxythiophene) (PEDOT) is a known conducting polymer exhibiting high conductivity, ranging from 10-2 to 103 S/cm. As PEDOT is insoluble in many common solvents, it is prepared by template polymerization with a polyanion, such as poly(styrene sulfonic acid) (PSSA). PSSA is a charge-balancing dopant during polymerization in water which allows for the formation of a colloidal dispersion of poly(3,4-ethylenedioxythiophene)-poly(styrenesulfonic acid) or PEDOT-PSS. PEDOT-PSS is commercially available and has desirable properties, such as high stability in the p-doped form, high conductivity, good film formation, and excellent transparency in the doped state. PEDOT-PSS dispersed in water can be spin-coated to result in transparent films.

The template polymer is typically a polyanion, comprising suitable functional groups to be a counterion to the conducting polymer. Suitable functional groups include sulfonic acid, phosphonic acid, and the like, or a combination thereof. The deprotonated sulfuric acid (sulfonate) serves as the negative ion to counterbalance the positive charge carrier on PEDOT.

Other conducting polymers include the conducting polymer-sulfonated poly(imide) complexes and conducting polymer-sulfonated poly(amic acid) complexes described in U.S. Pat. No. 8,753,542B2 to Sotzing which is incorporated by reference herein in its entirety.

The conducting polymer coating comprises a conducting polymer:template polymer film disposed on the stretchable insulating substrate comprising surface nucleophile derivatized nanoparticles. The conducting polymer:template polymer film can be cast onto the surface of the substrate comprising surface nanoparticles from solutions or dispersions comprising the conducting polymer:template polymer and optionally a surfactant in a suitable solvent using techniques known in the art. Suitable solvents for forming a cast film of conducting polymer:template polymer film depends upon the material. The solvent can be an organic solvent or combination of an organic solvent and water, specifically deionized water. Exemplary organic solvents include dichloromethane (DCM), dimethyl sulfoxide (DMSO), toluene, N,N-dimethyl formamide (DMF), propylene glycol monomethyl ether acetate (PGMEA), propylene glycol monomethyl ether (PGME), acetone, methanol, ethanol, tetrahydrofuran (THF), dimethylacetamide (DMAC), ethyl acetate and trifluoroacetic acid.

Suitable casting or coating processes to form the conducting polymer:template polymer film include drop casting, spin coating, ink jetting, spray coating, dip coating, flow coating, dye casting and the like, or a combination thereof. In one embodiment, the conducting polymer:template polymer film substantially covers a portion of a surface of the stretchable insulating substrate comprising surface nanoparticles, and specifically covers the entire surface. In another embodiment, the conducting polymer:template polymer film is applied to the surface of the stretchable insulating substrate comprising surface nanoparticles in the form of a pattern of any design. Exemplary patterning can be achieved by jetting or a waxing process (wax template), and the like.

After the conducting polymer:template polymer coating has been applied to the surface of the stretchable insulating substrate comprising surface nanoparticles solvent can be removed, if used, and the coating can be annealed. The annealing can be conducted at temperatures of about 80 to about 130° C., specifically about 90 to about 125° C., and yet more specifically about 100 to about 120° C. for as long as needed. Such conditions can be carried out in an oven or other suitable apparatus with or without vacuum or air flow.

The thickness of the conducting polymer:template polymer film can be about to about 40 nm to about 1 micrometer, specifically about to about 80 nm to about 500 nm, and more specifically about 100 nm to about 300 nm.

In an embodiment, the stretchable electrically conductive structure further comprises a conductive organic particle. In an embodiment, the conductive organic particle can be disposed between the stretchable insulating substrate and the conducting polymer:template polymer coating.

The conductive organic particle can be graphene, graphite, a combination of graphene and graphite, carbon nanotubes, buckyballs, “n-type” small molecules, or a combination thereof. Exemplary “n-type” small molecules include those commercially available from Sigma-Aldrich, including 2,9-bis[2-(4-chlorophenyl)ethyl]anthra[2,1,9-def:6,5,10-d′e′f′]diisoquinoline-1,3,8,10(2H,9H)tetrone; N,N′-bis(2,5-di-tert-butylphenyl)-3,4,9,10-perylenedicarboximide; 2,9-bis[2-(4-fluorophenyl)ethyl]anthra[2,1,9-def:6,5,10-d′e′f′]diisoquinoline-1,3,8,10(2H,9H)tetrone; 2,9-bis[(4-methoxyphenyl)methyl]anthra[2,1,9-def:6,5,10-d′e′f′]diisoquinoline-1,3,8,10(2H,9H)tetrone; N,N′-bis(3-pentyl)perylene-3,4, 9,10-bis(dicarboximide); 5,5′″-bis(tridecafluorohexyl)-2,2′:5′,2″:5″,2′″-quaterthiophene; 2,2′″-bis[4-(trifluoromethyl)phenyl]-5,5′-bithiazole; 5,10,15,20-tetraphenylbisbenz[5,6]indeno[1,2,3-cd:1′,2′,3′-lm]perylene; 2,9-diheptylanthra[2,1,9-def:6,5,10-d′e′f′]diisoquinoline-1,3,8,10(2H,9H)tetrone; 2,7-dihexylbenzo[lmn][3,8]phenanthroline-1,3,6,8(2H,7H)-tetrone; 4-(2,3-dihydro-1,3-dimethyl-1H-benzimidazol-2-yl)-N,N-dimethylbenzenamine; 4-(1,3-dimethyl-2,3-dihydro-1H-benzoimidazol-2-yl)-N,N-diphenylaniline; N, N′-dimethyl-3,4,9,10-perylenedicarboximide; N,N′-dioctyl-3,4,9,10-perylenedicarboximide; N,N′-dipentyl-3,4,9,10-perylenedicarboximide; [6.6] Diphenyl C62 bis(butyric acid methyl ester); N,N′-diphenyl-3,4,9,10-perylenedicarboximide; 2,9-dipropylanthra[2,1,9-def:6,5,10-d′e′f′]diisoquinoline-1,3,8,10(2H,9H)tetrone; N,N′-ditridecylperylene-3,4,9,10-tetracarboxylic diimide; [5,6]-Fullerene-C70; Fullerene-C60; Fullerene-C84; 1′,1″,4′,4″-tetrahydro-di[1,4]methanonaphthaleno[1,2:2′,3′,56,60:2″,3″][5,6]fullerene-C60; 1′,4′-Dihydro-naphtho[2′,3′:1,2][5,6]fullerene-C60; 1,4,5,8-naphthalenetetracarboxylic dianhydride; 1,2,3,4,5,6,7,8-octafluoro-9,10-bis[2-(2,4,6-trimethylphenyl)ethynyl]anthracene; perylene-3,4,9,10-tetracarboxylic dianhydride; [6,6]-phenyl-C61 butyric acid butyl ester; [6,6]-phenyl C61 butyric acid methyl ester; [6,6]-phenyl C71 butyric acid methyl ester; [6,6]-phenyl-C61 butyric acid octyl ester; 7,7,8,8-tetracyanoquinodimethane; 2,3,5,6-tetrafluoro-7,7,8,8-tetracyanoquinodimethane; 1,3,8,10(2H,9H)-tetraone, 2,9-bis(2-phenylethyl)anthra[2,1,9-def:6,5,10-d′e′f′]diisoquinoline; 1,3,6,8(2H,7H)-tetraone, 2,7-dicyclohexylbenzo[lmn][3,8]phenanthroline; [6,6]-thienyl C61 butyric acid methyl ester; and the like; or a combination thereof.

In an embodiment, the conductive organic particle used is graphene, graphite, or a combination of graphene and graphite to form a graphene and/or graphite infused stretchable substrate. Pristine graphene can be prepared by exfoliating pristine graphite via sonification in an organic solvent and water to yield graphene flakes. Exemplary organic solvents that can be used in the exfoliating process include alkyl (e.g. n-heptane) and aromatic (e.g. o-dichlorobenzene) solvents.

The total amount of conductive organic particle infused in the stretchable substrate can be about 0.2 to about 20 wt %, specifically about 1.0 to about 16 wt %, and more specifically about 2.5 to about 13 wt % based on the total weight of the conductive organic particle infused stretchable substrate. The total amount of graphene and/or graphite infused in the stretchable substrate can be about 0.2 to about 20 wt %, specifically about 1.0 to about 16 wt %, and more specifically about 2.5 to about 13 wt % based on the total weight of the conductive organic particle infused stretchable substrate.

In an exemplary embodiment, the conductive organic particle is graphene, graphite, or a combination of graphene and graphite infused, for example, by an interfacial trapping method to form a graphene and/or graphite infused stretchable insulating substrate. The interfacial trapping method generally involves exfoliating pristine graphite via sonification in an organic solvent and water to yield graphene flakes. Exemplary organic solvents that can be used in the exfoliating process include alkyl (e.g. n-heptane) and aromatic (e.g. o-dichlorobenzene) solvents. A stretchable insulating substrate is then exposed to the sonicated mixture and sonicated to infuse the graphene and/or graphite into the stretchable insulating substrate followed by removal of the substrate and drying to form a graphene and/or graphite infused stretchable insulating substrate. In general, the weight/volume ratio of graphite to organic solvent is about 20 mg/mL and the weight/volume ratio of graphite to organic and aqueous solvent is about 10 mg/mL.

The stretchable electrically conducing substrate in the form of a fiber can be used as a fiber, or at least two fibers can be woven, knitted, crocheted, knotted, pressed, or plied to form a multi-filament fiber or fabric. In one embodiment, a plurality of stretchable electrically conducing fibers can be used to manufacture a woven or nonwoven fabric. While these fabrics are generally in the form of a 2-dimensional woven or nonwoven planar sheet, their enhanced flexibility and stretchability permits them to be shaped into 3-dimensional conformations such as a rolled sheet, a folded sheet, a twisted sheet, a coiled sheet, or other configuration.

The term “fiber” as used herein includes single filament and multi-filament fibers, i.e., fibers spun, woven, knitted, crocheted, knotted, pressed, plied, or the like from multiple filaments. No particular restriction is placed on the length of the fiber, other than practical considerations based on manufacturing considerations and intended use. Similarly, no particular restriction is placed on the width (cross-sectional diameter) of the fibers, other than those based on manufacturing and use considerations. The width of the fiber can be essentially constant, or vary along its length. For many purposes, the fibers can have a largest cross-sectional diameter of 2 nanometers and larger, for example up to 2 centimeters, specifically from 5 nanometers to 1 centimeter. In an embodiment, the fibers can have a largest cross-sectional diameter of 5 to 500 micrometers, more particularly, 5 to 200 micrometers, 5 to 100 micrometers, 10 to 100 micrometers, 20 to 80 micrometers, or 40 to 50 micrometers. In one embodiment, the conductive fiber has a largest circular diameter of 40 to 45 micrometers. Further, no restriction is placed on the cross-sectional shape of the fiber. For example, the fiber can have a cross-sectional shape of a circle, ellipse, square, rectangle, or irregular shape.

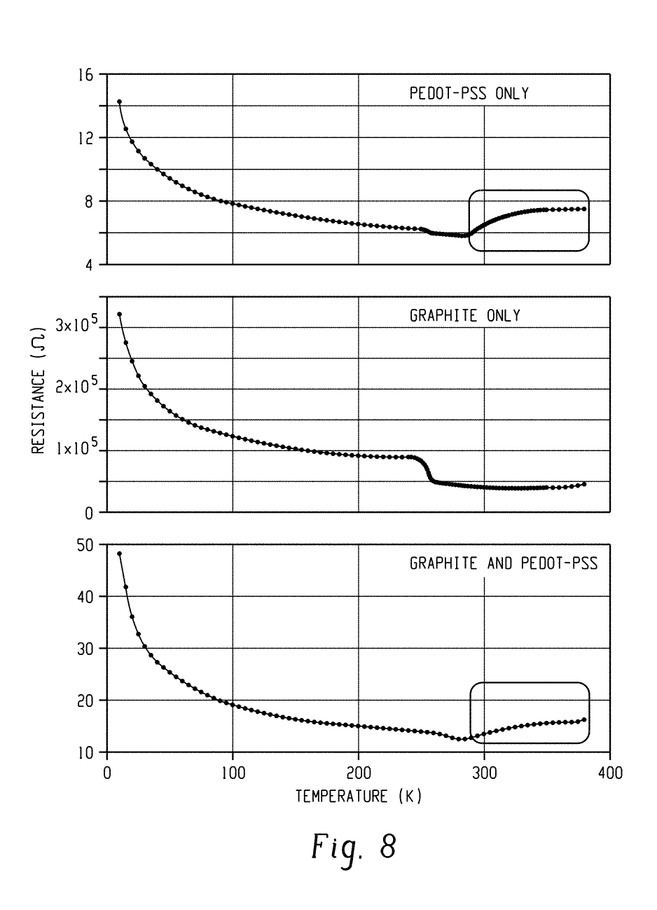

The stretchable electrically conductive structure can exhibit sheet resistance of about 1 to about 50 ohm/□ and more preferably 5 to about 20 ohm/□ wherein the resistance of the material will increase as a function of temperature thereby exhibiting metallic behavior at or approximately 270K. For example, FIG. 8 shows resistance versus temperature plot of a 4 wt. % PEDOT-PSS doped spandex fabric (top graph); a 7 wt % graphene/graphite infused spandex fabric (middle graph); and a 4 wt % PEDOT:PSS doped spandex fabric containing 7 wt % graphene/graphite (bottom graph). The grey box insets of the top and middle graphs shows metallic behavior on a stretchable spandex substrate. Additionally, the PEDOT:PSS coating does not crack when stretched when observed by optical microscopy.

The conductive polymer fabric electrodes or conductive polymer fabric wiring elements for use as the conductive element in pads for monitoring applications are suitable for use in dry environments, wet environments e.g. exposed to sweat, rain, under water, including fresh or salt water. For example, PEDOT-PSS infused fabrics demonstrated retention of properties after repeated washing cycles with excessive amounts of detergent and prolonged clothes dryer exposure (12 hours at 120° C.) for three complete cycles without loss of conductivity.

The monitoring system device can include one or more conductive polymer fabric electrodes or wiring elements, specifically two or more, and more specifically two.

The conductive polymer fabric electrodes or conductive polymer fabric wiring elements for use as the conductive element in pads for wearable monitoring systems find application in the sports industry, electronics industry, and healthcare/medicine industry. The monitoring pads comprising the conductive polymer fabric electrode or wiring elements as the conductive element can be prepared in any suitable size and shape for the particular application.

The electrodes used to measure bio-potentials as disclosed herein are free of Ag/AgCl sensors and free of other metals such as copper and silver. In certain embodiments, however, a metal wire (insulated copper electrical wires) or similar component can be used as a connector connecting the electrode to other components of the monitoring device (e.g. ECG device).

In an embodiment, a bio-potential wearable monitoring system comprises an electrode comprising a conductive polymer fabric, and a wiring element in electrical communication with the electrode; wherein the conductive polymer fabric is a.) an electrically conductive fibrous substrate comprising a fibrous substrate comprising polymeric fibers comprising nucleophile derivatized nanoparticles wherein a portion of the nucleophile derivatized nanoparticles are located at the surface of the polymeric fiber; and an electrically conductive polymer film disposed on at least a portion of the polymeric fibers of the fibrous substrate and at least in partial contact with the nucleophile derivatized nanoparticles; or b.) a stretchable electrically conductive structure comprising a stretchable insulating substrate comprising nucleophile derivatized nanoparticles located at the surface of the stretchable insulating substrate, wherein the stretchable insulating substrate is a fiber or fabric; and a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate. Within this embodiment, the wearable monitoring system is in the form of a garment, footwear, headwear, wrist band, chest strap, or belt; and the monitoring system is an electrocardiogram system, an electroencephalogram system, an electromyography system, heart rate system, respiratory rate system, bioelectrical impedance analysis system, or an electrodermal activity system. When the monitoring system is an electrocardiogram system, it can have a signal to noise ratio range of 3 to 30 dB; and be capable of measuring ECG in the heart rate range of 40 to 180 bpm while in the ‘wet’ (with lotion and/or hydrogel) condition. Further when the monitoring system is an electrocardiogram system, it can have a signal to noise ratio range of 3 to 30 dB; and be capable of measuring ECG in the heart rate range of 40 to 180 bpm while in the ‘dry’ (without lotion or hydrogel) condition.

The following illustrative examples are provided to further describe the invention and are not intended to limit the scope of the claimed invention.

### EXAMPLES

Example 1 Formation of a carbon black/polydimethylsiloxane pad electrodes with integrated conductive polymer fabric—use as electrodes for ECG monitoring systems

Pad electrodes prepared from carbon black/polydimethylsiloxane having integrated flexible or integrated flexible/stretchable conductive polymer fabrics as the connecting electrode. The integrated conductive polymer fabric electrodes were prepared using a modified procedure for the formation of carbon black filled polydimethylsiloxane ECG electrodes having integrated copper mesh as disclosed in Noh et al. “Novel Conductive Carbon Black and Polydimethlysiloxane ECG Electrode: A Comparison with Commercial Electrodes in Fresh, Chlorinated, and Salt Water” Annals of Biomedical Engineering published online 2016 Jan. 14. Noh et al. used the copper mesh to maximize transduction of the signal response from the carbon black (CB) filled polydimethylsiloxane (PDMS) that comes in contact with the skin of the individual. In the present Example, the conductive polymer fabric was used as the connecting electrode in place of the copper mesh of Noh et al.

Electrode patterns of various sizes and shapes were prepared. The conductive polymer fabric samples were prepared from a flexible polymeric fabric or a flexible and stretchable polymeric fabric infused or coated with a conductive polymer. The flexible fabric was polyethylene terephthalate (PET) synthetic leather, a melt processed nonwoven material, the fibers of which contained metal oxide nanoparticles (5 to 50 nanometers) embedded within and/or on the surface of the fibers.

The flexible/stretchable fabric was a PET/polyurethane knitted/woven spandex fabric available from Lubrizol the fibers of which contained metal oxide nanoparticles (5 to 50 nanometers) embedded within and/or on the surface of the fibers. Commercially available PEDOT-PSS was used as the conductive polymer: CLEVIOS PH1000, a poly(3,4-ethylenedioxythiophene):poly(styrenesulfonate) aqueous dispersion commercially available from Heraeus Clevios™ PH1000, 1-1.3% solids content, PEDOT:PSS ratio by weight=˜1:2.5, specific conductivity after addition of 5% dimethyl sulfoxide (DMSO) measured on the dried coating=850 S/cm.

Formation of the conductive polymer fabric was conducted by applying PEDOT PSS to the fabric samples either by brush stenciling, screen printing, or bulk infusion using procedures set out in U.S. Patent Publication No. 2015/0017421A1 to Sotzing.

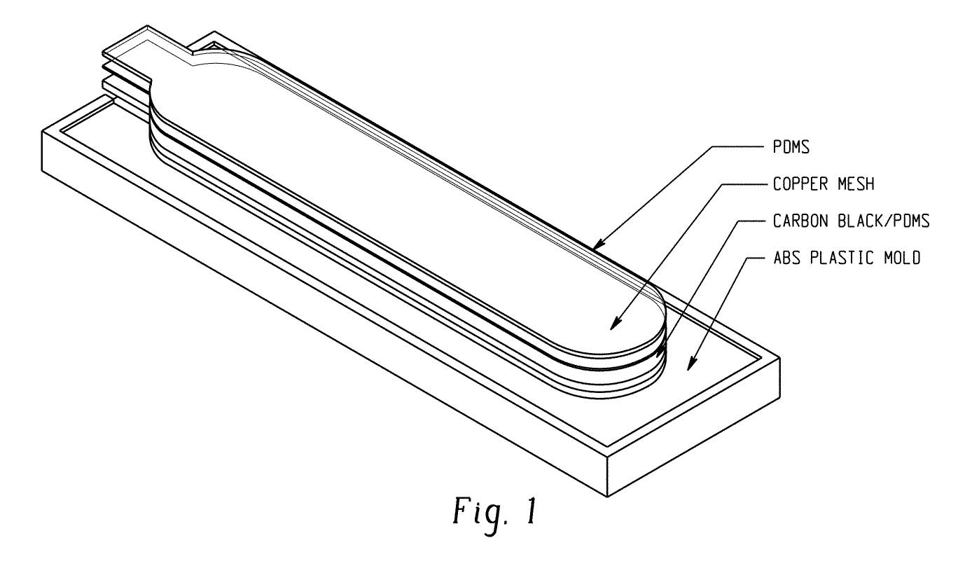

Each resulting conductive polymer fabric was used as the connecting electrode to replace copper mesh within the pads of a carbon black filled polydimethylsiloxane ECG electrode as disclosed in Noh et al. That is, the conductive polymer fabric was used as the current carrying source from the transducer to the processor. The integrated copper mesh electrode of Noh et al. was used as a Comparative electrode. FIG. 1 is an illustration of the electrode fabrication cavity mold and fabrication procedure showing the location of the copper mesh of the Comparative electrode. In Step 1 of the fabrication, an acrylonitrile butadiene styrene (ABS) cavity mold was filled with the conductive CB/PDMS composite and leveled such that no excess material remains. Step 2, a copper mesh with an attachment point (Comparative) was then affixed on the CB/PDMS mix to allow signal acquisition via the monitoring device. Specifically, an insulated and waterproofed wire was soldered to the embedded mesh, and used as a connector to an ECG monitoring device. In Examples 1A-1F, the copper mesh was replaced with a conductive polymer fabric. Step 3: A PDMS and curing agent mixture was then used to encapsulate the exposed surface with embedded copper mesh or embedded conductive polymer fabric. Step 4 involved degassing all components for an additional 15 minutes in a vacuum chamber. Step 5 the fasteners were soldered to the exposed end of the wire extending from the electrode. Step 6 the completed electrode assembly was then placed in a curing oven at 75° C. for 3 hours. Step 7 after the 3 hours, the molds were removed from the curing oven and subsequently the electrodes were also removed from the cavity molds.

Table 1. lists the electrodes used in the study to determine the effectiveness of CB/PDMS electrode pads having different types of integrated conductive polymer fabric as ECG electrode pads compared to a CB/PDMS electrode pad having integrated copper mesh. ECG measurements were conducted using similar procedures as disclosed in Noh et al.

TABLE 1

| | Electrode Pad | |
|---|---|---|
| ECG electrode | Material | Embedded Current Carrier |
| A | CB/PDMS | Brush coated PEDOT-PSS/PET |
| | | synthetic leather |
| B | CB/PDMS | Screen printed PEDOT-PSS/PET |
| | | synthetic leather |
| C | CB/PDMS | Bulk infused PEDOT-PSS/PET |
| | | synthetic leather |
| D | CB/PDMS | Brush coated PEDOT-PSS/ |
| | | PET/polyurethane spandex fabric |
| E | CB/PDMS | Screen printed PEDOT-PSS/ |
| | | PET/polyurethane spandex fabric |
| F | CB/PDMS | Bulk infused PEDOT-PSS/ |
| | | PET/polyurethane spandex fabric |
| Comparative | CB/PDMS | Copper mesh |
| (Noh et al.) | | |

Use of the conductive polymer fabric as the connecting electrode in the electrocardiogram application, regardless of the method used to apply the conductive polymer to the fabric (e.g. brush coated conductive synthetic leather Example A, screen-printed conductive synthetic leather Example B, or completely soaked (bulk infusion) conductive synthetic leather Example C), has been shown to give responses that match those of the copper mesh integrated Comparative electrode.

The results of the study revealed the ability to replace the integrated copper mesh used in the Comparative ECG pad to maximize the signal from the transduction element, in this case the carbon black conductors imbedded in the polydimethylsiloxane matrix. It is envisioned that the PEDOT-PSS conductive polymer fabric electrodes of Example 1 can be applied to any pad of a similar nature having a conductor within an insulating matrix of a higher resistivity than the PEDOT-PSS conductive polymer fabric.

### Example 2. Electrically Conductive Polyethylene Terephthalate Synthetic Leather—PEDOT:PSS, Comparison of Finished and Unfinished Synthetic Leather

Samples of finished (material having a glossy surface) and unfinished (material without a glossy surface) polyethylene terephthalate (PET) synthetic leather were prepared into electrically conductive synthetic leather. Samples of the finished synthetic leather were cut to 1.5×1 inch swabs. Samples of the unfinished synthetic leather were cut to 2.0×1.5 inch swabs. The electrically conductive polymer used was Clevios™ PH1000 (Poly(3,4-ethylenedioxythiophene):poly(styrenesulfonate) aqueous dispersion (PEDOT:PSS, Clevios™ PH1000 commercially available from Heraeus as an aqueous dispersion, 1-1.3% solids content, PEDOT:PSS ratio by weight=˜1:2.5, specific conductivity after addition of 5% dimethyl sulfoxide (DMSO) measured on the dried coating=850 S/cm) or Clevios™ P (Clevios™ P aqueous dispersion of PEDOT:PSS commercially available from Heraeus, 1.2-1.4% solids content). The application method was drop casting and 2 point resistances were measured (4 line measurements can also be used wherein current is measured between the outer leads, and voltage drop measured between the inner leads). Table 2 contains a summary of the samples, electrically conductive polymer used, method of applying the electrically conductive polymer, and the results of resistance measurements.

TABLE 2

| | Electrically | | |
|---|---|---|---|
| Sample, | conductive | | Resistance |
| type | polymer | Application method | measurement |
| 2-1 finished | Clevios ™ | Drop cast, allowed to stand 10 | >10 MOhm/square |
| | PH1000 | minutes and then the sample was | |
| | | rinsed with water | |
| 2-2 finished | Clevios ™ | Drop cast ~1.5 ml, water removed | 3 kOhm/square |
| | PH1000 | by heating 70° C. for 10 minutes | |
| 2-3 finished | Clevios ™ P | Drop cast ~1.5 ml, water removed | 15 kOhm/square |
| | | by heating 70° C. for 10 minutes | |
| 2-4 finished | Clevios ™ P | Flow coated to create a thickness | 40 kOhm/square = |
| | | gradient of electrically conductive | thickest region of |
| | | polymer, water removed by heating | coating, most |
| | | 70° C. for 10 minutes | conductive |
| | | | 1 MOhm/square = thin |
| | | | region of coating |
| 2-5 | Clevios ™ | Drop casting ~2.0 ml wicked into | 4.5 kOhm/square (top |
| unfinished | PH1000 | sample, water removed by heating | side*) |
| | | 70° C. for 1 hour | 8 kohm/square (bottom |
| | | | side**) |
| 2-6 | Clevios ™ | Drop casting ~2.0 ml wicked into | 45 Ohm/square (top |
| unfinished | PH1000 | sample, water removed by heating | side*) |
| | with 5 wt % | 70° C. for 1 hour | 100 Ohm/square |
| | DMSO | | (bottom side**) |

*top side is the side of the sample where the electrically conductive polymer was cast.
**bottom side is the opposite side of the sample where the electrically conductive polymer was cast.

Sample 2-1 was prepared to determine if the Clevios™ PH1000 absorbed into the finished leather. No wicking behavior of the electrically conductive polymer dispersion was observed for the finished synthetic leather samples. However, unoptimized resistances of 3 kOhm can be achieved with films cast atop the glossy surface of the finished synthetic leather samples. Using 5 wt % DMSO results in a resistance of 20 Ohms, two orders of magnitude better than films prepared without DMSO.

The unfinished samples showed wicking behavior through the thickness of the synthetic leather sample, with the top side containing a higher concentration of electrically conductive polymer compared to the bottom side. The electrically conductive polymer wicked the dispersion radially from the application point. The wicking behavior is likely due to the more porous nature of the unfinished synthetic leather compared to finished synthetic leather.

Using Clevios™ PH1000 with 5 wt % DMSO on the unfinished leather resulted in a phenomenal low resistance measurement of 45 Ohms/square. With further optimization resistance measurement as low as 0.4 Ohms/square have been achieved. It should be noted that good quality Indium doped tin oxide (ITO) coatings on polyethyleneterephthalate (PET) have 60 Ohms/square sheet resistance with the absolute best being at around 15 Ohms/square. ITO on glass has its best sheet resistance between 8 and 10 Ohms/square. Side by side comparisons with ITO on glass and ITO on plastic were conducted, in addition to electrical breakdown measurements on the ITO compared to the conductive leather showing that the conductive leather can handle higher current and power before breakdown. On the unoptimized samples, ITO coated PET-quality conductivity can be achieved with the unfinished synthetic leather. A lower resistance could be achieved through modification of the heat treatment.

### Example 3. Electrically Conductive Polyethylene Terephthalate Synthetic Leather—PEDOT:PSS Percolation 110° C.

PEDOT:PSS aqueous dispersion Clevios™ PH1000 was applied in various concentrations to synthetic leather (polyethylene terephthalate-based), the fibers of which contain SiO₂ nanoparticles (˜50 nanometers) embedded within and on the surface of the fibers. Sample preparation involved soaking the synthetic leather (PET) once and dried once. DMSO (5 wt %) in Clevios™ PH1000 was drop-coated onto all samples, with dilutions of the PEDOT:PSS to get values below 2% by weight. Resistance (Ohm) was measured using a sample holder with fixed silver probes ¾ inches apart and the results are provided in Table 3.

TABLE 3

| | % weight of DMSO and | |
|---|---|---|
| Sample | PEDOT:PSS | Resistance (Ω) |
| 1 | 0.5 | 216 |
| 2 | 0.98 | 44.3 |
| 3 | 1.25 | 22.4 |
| 4 | 1.97 | 16 |
| 5 | 2.87 | 14.2 |

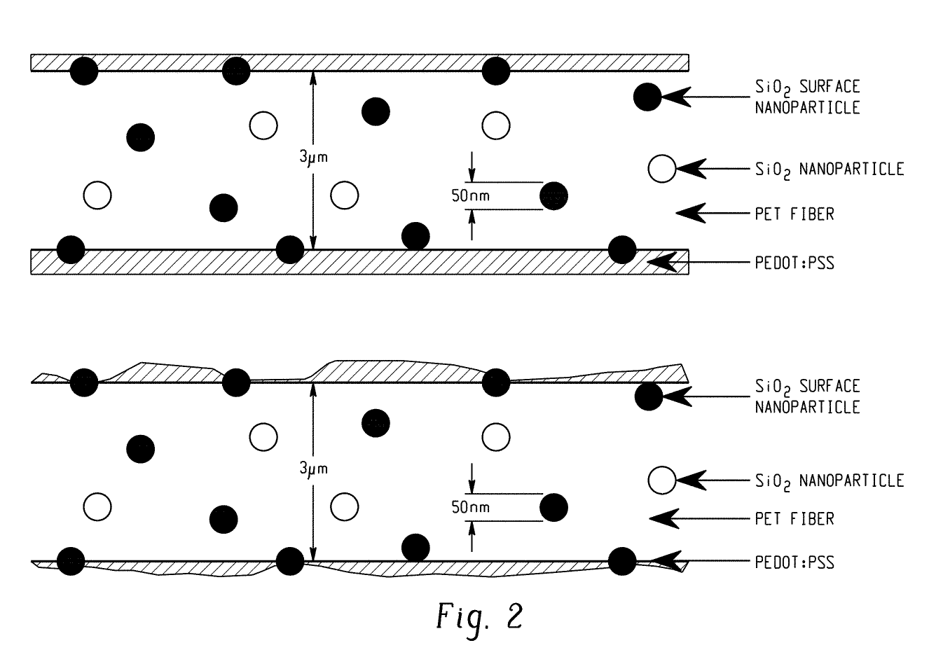

A generalized schematic of a PET fiber of the synthetic leather is shown in FIG. 2 where there are nanoparticles of SiO₂ embedded in the fiber (dotted circles) and located at the surface of the fiber (dark circles). The SiO₂ nanoparticles located at the surface of the fiber are in contact with the PEDOT:PSS coating.

### Example 4. Electrically Conductive Synthetic Leather—PEDOT

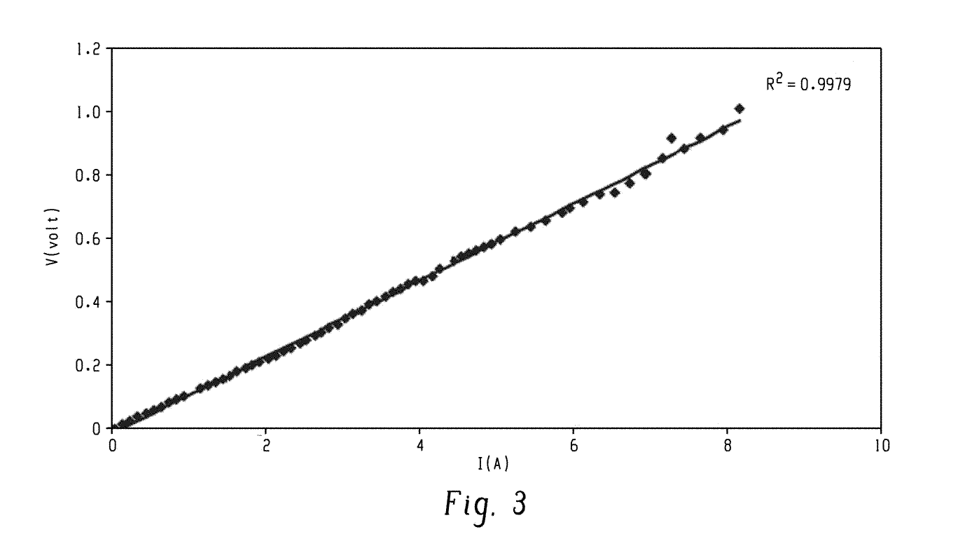

PEDOT:PSS was applied to synthetic leather (polyethylene terephthalate-based) which comprises nanoparticles of SiO2. The sample was capable of passing 8 amps of current. The ohmic behavior of the sample is shown in FIG. 3 which is a plot of the current in ampere (I (A)) versus voltage (V) in volts. The R2=0.9979 and the resistance=0.117Ω.

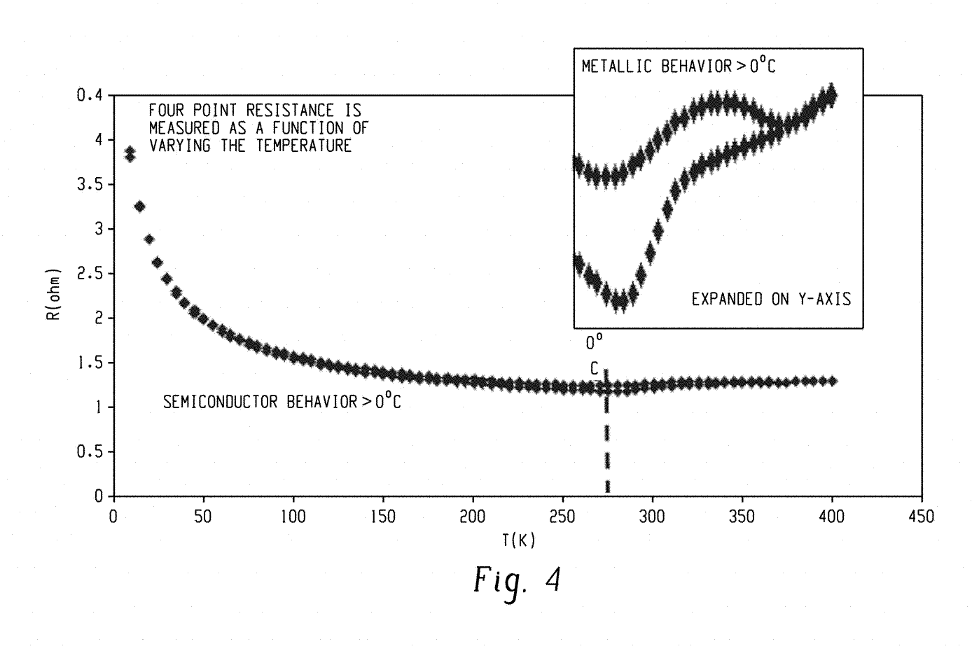

The sample further shows temperature dependent behavior. Four point resistance is measured as a function of varying the temperature and plotted as Temperature in Kelvin (T(K)) versus Resistance (R(Ohm)) in FIG. 4. As shown in the graph, the sample exhibited semiconductor behavior at temperatures less than 0° C. (273.15 K). However, unexpectedly, the material exhibits metallic behavior at temperatures above 0° C. (273.15 K) as shown in the expanded plot insert in FIG. 4.

### Example 5. Comparison Between Synthetic Leather Substrates, with and without SiO₂ Desiccant Particles

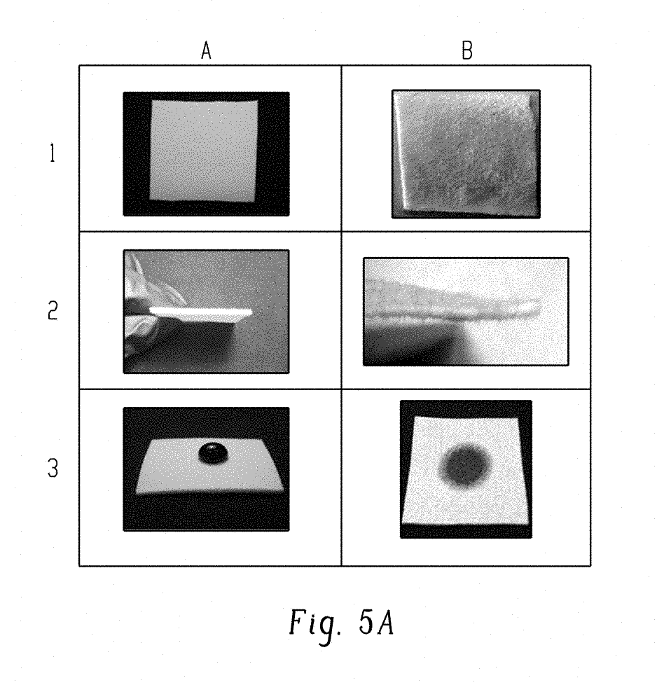

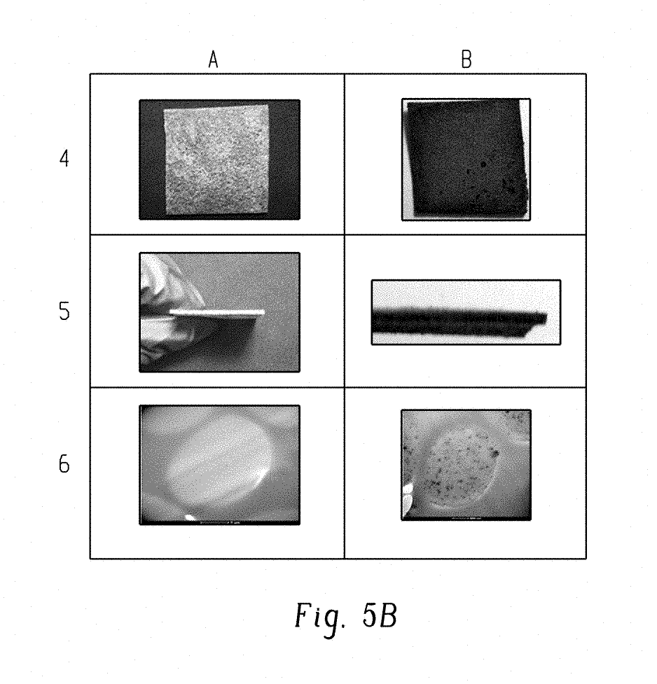

FIG. 5A and FIG. 5B include a series of images illustrating the differences between electrically conductive synthetic leather prepared from synthetic leather free of desiccant particles (Column A) and synthetic leather containing desiccant particles (Column B).

Column A: White nonwoven PET unfinished synthetic leather without desiccant nanoparticles.

Column B: Grey nonwoven PET unfinished synthetic leather with SiO₂ nanoparticles having an average diameter of 100 nm with a range in diameter size from a lower limit of 50 and upper limit of 150 nm.

A1: Top surface view of leather without nanoparticles, 1 inch×2 inch sample size.

A2: Side view of leather without nanoparticles, thickness 0.8 mm.

A3: 5 wt. % DMSO doped PEDOT:PSS droplet on top surface of leather without nanoparticles.

A4: Clevios™ PH1000 was doped with 5% by weight DMSO. The solution does not wick into the PET leather without nanoparticles, therefore the sample was soaked for 15 minutes. The leather was dried at 110° C. for one hour and the weight percent of doped PEDOT:PSS remaining on the dried white PET is 6.8%. The resistance on the top surface as measured by two point probe at ¾″ ranged from 20-40 Ohms. The resistance on the bottom ranged from 30-50 Ohms.

A5: Side view of white PET leather not containing nanoparticles with PEDOT-PSS, after annealing at 110° C.

A6: Transmission electron microscopy (“TEM”) image of PET leather not containing nanoparticles, without PEDOT:PSS.

B1: Top surface view, 1 inch×2 inch sample size. White nonwoven PET unfinished synthetic leather with SiO₂ nanoparticles.

B2: Side view of white nonwoven PET unfinished synthetic leather with nanoparticles, thickness 0.6 mm.

B3: 5 wt % DMSO doped PEDOT:PSS droplet on top surface of white unfinished synthetic PET leather containing nanoparticles.

B4: Top view of unfinished PET 5.7 wt % PEDOT:PSS leather and annealing temperature 110° C. for 1 hour; R=0.548 Ohm/sq and maximum current 3.2 A before breakdown.

B5: Side view of PEDOT-PSS treated white PET containing SiO₂ nanoparticles, thickness 0.8 mm.

B6: TEM image of untreated PET leather containing SiO₂ nanoparticles without PEDOT:PSS; the nanoparticles are clearly visible in the image.

### Example 6. Electrically Conductive Polyethylene Terephthalate Electrospun Nanofiber Mat with and without Silica

Polyethylene terephalate (PET) electrospun fibers and PET/silica electrospun fibers were individually prepared as free standing mats having densities of about 0.1 g/ml. PET/silica solution was prepared by dissolving PET solution in 50:50 trifluoroacetic acid:dichloromethane. Hydrophilic fumed silica is added and the resulting mixture is shear mixed for 15 minutes to result in a 20 weight PET+3 weight silica solution. Electro-spinning of the solution was conducted at a flow rate of 3 milliliters (ml)/hour, applied potential: 15 kV, collector plate distance: 15 cm, and run time: 4 or 8 hours to form a fiber mat.

PET mats without silica were also prepared using the same process.

After electrospinning, the fiber mats were dried at 75° C. in a drying oven for 30 minutes. The dried fiber mats were then plasma treated to provide good wettability and adhesion of PEDOT-PSS on the surface of the PET or PET/silica fiber mat. Plasma etching was performed using a Fischione instruments model 1020 plasma cleaner with a 13.56 MHz oscillating field system. Operating parameters were as follows; pull vacuum for 2-3 minutes (once instrument indicates a ready-state) while flowing 25%/75% oxygen:argon, apply plasma for 5 seconds.

The mats were infused with PEDOT-PSS+5 wt % DMSO by using either a drop casting method (“drop cast method”) or by completely submersing the mat in diluted PEDOT-PSS+5 wt % DMSO solution (PEDOT-PSS: 1 gram, Water: 4 grams, DMSO: 50 milligrams) (“soaking method”). After 30 minutes the mats were removed from the PEDOT-PSS+5 wt % DMSO mixture and allowed to air dry for 30 minutes. The mats were then annealed for 1 hour at 110° C. in air.

Conductivity measurements of the sheets were conducted using a four-line silver paint contact to measure voltage as a function of current. A minimum of 10 voltage/current data points were taken to plot I-V at room temperature. Sheet resistance was calculated based on the slope of the curve-(Keithley224 Programmable was used as power source). Resistivity was calculated based on film thickness measurements done by scanning electron microscopy (SEM). The results are provided in Table 4.

TABLE 4

| | | PEDOT-PSS | Rs |
|---|---|---|---|
| | Run | loading | (ohms/ |
| Formulation | time | (% wt) | square) |
| 20 wt PET with silica; drop cast | 8 hours | 3.46 | 4 |
| PEDOT-PSS | | | |
| 20 wt PET without silica; drop cast | 4 hours | 2.79 | 20 |
| PEDOT-PSS | | | |
| 20 wt PET with silica; drop cast | 4 hours | 3.75 | — |
| PEDOT-PSS | | | |
| 20 wt PET with 3 wt silica; | 8 hours | 2.16 | 4.88 |
| PEDOT-PSS soaking method | | | (average) |

### Example 7. Formation of a Stretchable Organic Metal: PEDOT:PSS, No Stretch

One inch by one inch square sample of stretchable insulating fabric of 85% nylon 15% Spandex containing 3% surface area silica nanoparticles (according to X-ray photoelectron spectroscopy (“XPS”)) was soaked in two cycles of PEDOT:PSS (CLEVIOS PH1000, an aqueous dispersion of poly(3,4-ethylenedioxythiophene)-poly(styrenesulfonate), PEDOT:PSS ratio 1:2.5 (by weight) and a solid content of 1.0-1.3%, commercially available from Heraeus CLEVIOS GmbH) (95 wt %)+dimethyl sulfoxide (DMSO) (5 wt %) to achieve a weight percentage of PEDOT:PSS of about 6.02%. Each soaking cycle involved placing the fabric in a container and dropping the PEDOT:PSS on the top until the fabric was saturated. The top side of the fabric sample is referred to as “FRONT”. The resulting sample was annealed at 110° C. for 1 hour and then hung to dry.

Sheet resistance was measured for the annealed samples. All resistances were calculated from an I-V curve at room temperature, with a minimum of 10 data points. Electrical data was obtained using a four line probe fabricated in house according to literature design with grooves carved for the leads ensuring a uniform length and spacing was obtained. [R. K. Hiremath, M. K. Rabinal, B. G. Mulimani, Rev. Sci. Instrum. 2006, 77, 126106.] Current was passed through the outer two electrodes, while the inner two measured voltage. Sheet resistance was calculated based on the relationship Rs=R(w/l) where w is the width of the sample (2.5 cm), and 1 is the distance between the leads (0.35 cm). [Hiremath, 2006] Two current sources were used, a Keithley 224 Programmable for small current (I max=101.1×10-3 A), Power Supply 3630 for high current I max=10 A, and a 196 system DMA was used to measure the voltage. The results are shown in Table 5.

TABLE 5

| | Sheet Resistance (Ω/sq) |
|---|---|
| Front - | 2.651 |
| Front \| | 6.676 |
| Back - | 7.455 |
| Back \| | 4.153 |

### Example 8. Formation of a Stretchable Organic Metal: PEDOT:PSS, Preliminary Stretch

One inch by one inch square samples of stretchable insulating fabric of 90% polyester 10% Spandex having 3% surface area silica nanoparticles (according to XPS) were soaked under stretch in two cycles of PEDOT:PSS (CLEVIOS PH1000) (95 wt %)+DMSO (5 wt %). A first sample was soaked under a 2-way stretch (120%) to achieve a weight percentage of PEDOT:PSS of about 7.91%. A second sample was soaked under a 4-way stretch (120%) to achieve a weight percentage of PEDOT:PSS of about 5.54%. As in Example 7, each soaking cycle involved placing the fabric in a container and dropping the PEDOT:PSS on the top until the fabric was saturated, however for Example 8 the samples were soaked while stretched. The top side of the fabric sample is referred to as “FRONT”. The resulting samples were annealed at 110° C. for 1 hour under stretch.

Sheet resistance was measured for the annealed samples as set out in Example 7; the results are shown in Table 6.

TABLE 6

| | Sheet Resistance (Ω/sq) | Sheet Resistance (Ω/sq) |
|---|---|---|
| | 2-way stretch 120%, | 4-way stretch 120%, |
| | 7.91% PEDOT:PSS | 5.54% PEDOT:PSS |
| Front - | 12.50 | 10.41 |
| Front \| | 34.55 | 20.22 |
| Back - | 36.08 | 18.33 |
| Back \| | 14.57 | 17.17 |

### Example 9. Formation of a Stretchable Organic Metal: PEDOT:PSS, Preliminary Stretch

A one inch by one inch square sample of stretchable insulating fabric of 90% polyester 10% Spandex having 3% surface area silica nanoparticles (according to XPS) was soaked under stretch in two cycles of PEDOT:PSS (CLEVIOS PH1000) (95 wt %)+DMSO (5 wt %) to achieve a weight percentage of PEDOT:PSS of about 5.23%. As in Example 7, each soaking cycle involved placing the fabric in a container and dropping the PEDOT:PSS on the top until the fabric was saturated, however for Example 9 the sample was soaked while stretched. The top side of the fabric sample is referred to as “FRONT”. The resulting sample was annealed at 110° C. for 1 hour and allowed to relax during annealing and then hung to dry.

Sheet resistance was measured on the annealed sample as set out in Example 7; the results are shown in Table 7.

TABLE 7

| | Sheet Resistance (Ω/sq) |
|---|---|
| Front—(stretch direction) | 2.592 |
| Front \| | 8.245 |
| Back - | 6.676 |
| Back \| | 3.474 |

### Example 10. Formation of a Stretchable Organic Metal: PEDOT:PSS; Plasma Treatment

A one inch by one inch square sample of stretchable insulating fabric of 90% polyethylene terephthalate 10% Spandex having 3% surface area silica nanoparticles (according to XPS) was plasma treated for five seconds and then soaked in one cycle of PEDOT:PSS (CLEVIOS PH1000) (95 wt %)+DMSO (5 wt %) to achieve a weight percentage of PEDOT:PSS of about 3.63% after annealing for 1 hour at 110° C. The resulting PEDOT:PSS film thickness was 204±167 nm as determined by scanning electron microscopy (SEM). The sample exhibited a sheet resistance from four-probe measurement of 17.17±3.53 Ω/sq. All resistances were calculated from an I-V curve at room temperature, with a minimum of 10 data points. Electrical data was obtained using a four line probe fabricated in house according to literature design with grooves carved for the leads ensuring a uniform length and spacing was obtained. [R. K. Hiremath, M. K. Rabinal, B. G. Mulimani, Rev. Sci. Instrum. 2006, 77, 126106.] Current was passed through the outer two electrodes, while the inner two measured voltage. Sheet resistance was calculated based on the relationship Rs=R(w/l) where w is the width of the sample (2.5 cm), and 1 is the distance between the leads (0.35 cm). [Hiremath, 2006] Two current sources were used, a Keithley 224 Programmable for small current (I max=101.1×10-3 A), Power Supply 3630 for high current I max=10 A, and a 196 system DMA was used to measure the voltage.

A similar sample of 90% PET 10% Spandex stretchable insulating fabric having 3% surface area silica nanoparticles (according to XPS) was plasma treated for twenty seconds and then soaked in two cycles of PEDOT:PSS (CLEVIOS PH1000) (95 wt %)+DMSO (5 wt %) to achieve a weight percentage of PEDOT:PSS of about 3.84% after annealing for 1 hour at 110° C. after each soak. The sample exhibited a sheet resistance from a four-probe measurement of 14.36±2.89 Ω/sq.

### Example 11. Formation of a Stretchable Organic Metal: PEDOT:PSS; Plasma Treatment

A one inch by one inch square sample of stretchable insulating fabric of 85% nylon 15% Spandex having 3% surface area silica nanoparticles (according to XPS) was plasma treated for five seconds and then soaked in one cycle of PEDOT:PSS (CLEVIOS PH1000) (95 wt %)+DMSO (5 wt %), annealed at 110° C. for 1 hour to achieve a weight percentage of PEDOT:PSS of about 4.40%. The resulting PEDOT:PSS film thickness was 225±106 nm as determined by scanning electron microscopy (SEM). The sample exhibited a sheet resistance from four-probe measurement of 12.21±0.72 Ω/sq.

A similar sample of 85% nylon 15% Spandex stretchable insulating fabric having 3% surface area silica nanoparticles (according to XPS) was plasma treated for sixty seconds and then soaked in two cycles of PEDOT:PSS (CLEVIOS PH1000) (95 wt %)+DMSO (5 wt %), annealed at 110° C. for 1 hour to achieve a weight percentage of PEDOT:PSS of about 6.96%. The sample exhibited a sheet resistance from a four-probe measurement of 4.80±0.85 Ω/sq.

### Example 12. Formation of a Stretchable Organic Metal: PEDOT:PSS, Graphene; and Comparative Stretchable Fabric with Graphene Alone

A one inch by one inch square sample of stretchable insulating fabric of 90% PET 10% Spandex having 3% surface area silica nanoparticles (according to XPS) was plasma treated for twenty seconds, sonicated in graphite/heptane/water for 1 hour, dried in an over, and then soaked in two cycles of PEDOT:PSS (CLEVIOS PH1000) (95 wt %)+DMSO (5 wt %), annealed at 110° C. for 1 hour to achieve a weight percentage of graphite of 5.96% and PEDOT:PSS of 7.15%. The sample exhibited a sheet resistance from four-probe measurement of 3.02 Ω/sq.

A comparative sample of 90% PET 10% Spandex stretchable insulating fabric having 3% surface area silica nanoparticles (according to XPS) was plasma treated for twenty seconds sonicated in graphite/heptane/water for 1 hour, dried in an over to achieve a weight percentage of conductor of about 9.12%. The sample exhibited a sheet resistance from a four-probe measurement of 9071.43 Ω/sq.

### Example 13. Phase Segregation of PEDOT and PSS from the Outer Surface of PEDOT:PSS Films

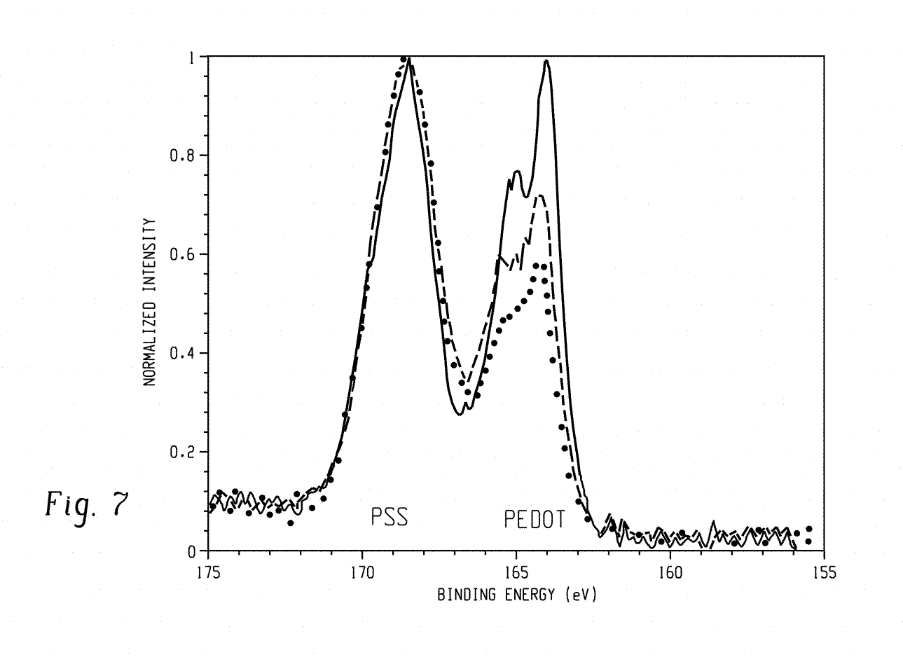

The phenomenon of PEDOT and PSS phase segregation as observed on the outer surface of the PEDOT-PSS films was analyzed by x-ray photoelectron spectroscopy (XPS). FIG. 7 discloses three XPS traces for PEDOT-PSS films on (dotted line) a control sample of electrospun PET fibers of 3 micrometer diameter with 150 nm thick PEDOT-PSS film, (dashed line) PET electrospun mat of same fiber diameter having silica nanoparticles with 150 nm thick film of PEDOT-PSS, and (solid line) PEDOT-PSS film of 130 nm thickness on synthetic leather fibers having silica nanoparticles. As shown in FIG. 7, the two bands between 162 and 166 eV are the spin-split doublet S(2P), S(2p1/2) and S(2p3/2), bands from the sulfur in PEDOT. [X. Crispin, S. Marciniak, W. Osikowicz, G. Zotti, A. W. D. van der Gon, F. Louwet, M. Fahlman, L. Groenendaal, F. De Schryver, W. R. Salaneck, J. Polym. Sci. Part B Polym. Phys. 2003, 41, 2561; U. Voigt, W. Jaeger, G. H. Findenegg, R. v. Klitzing, J. Phys. Chem. B 2003, 107, 5273] The energy splitting is ˜1.2 eV, the respective intensities have a ratio of 1:2 and the components typically have the same full width at half maximum and shape. In the case of the sulfur S2p from PSS, the binding energy bands are found at higher energy between 166 and 172 eV. The broad peak is composed of the spin-split doublet peaks. This broadening effect is due to the sulfonate group existing in both the neutral and anionic state. Therefore, there is a broad distribution of different energies in this high molecular weight polymer. The same applies to PEDOT although the number of charged and neutral species are not as large in number. The PEDOT:PSS ratio was calculated by measuring the integral area ratio of peaks assigned to PEDOT and PSS. The ratio of PEDOT to PSS increased from 1 to 1.95 for the control consisting of PET fibers without silica having a coating of PEDOT-PSS to a ratio of 1 to 1.2 for PET fibers containing silica nanoparticles translating to an 80% reduction of PSS at the surface. The PEDOT to PSS ratio of 1 to 1.95 of the control sample consisting of PEDOT-PSS film coated on PET fibers without silica agrees well with the manufacture specifications for the Clevios PH1000 [Coating Guide Clevios™ P Formulations. 1-12 (2012) at http://www.heraeus-clevios.com/media/webmedia local/media/datenblaetter/Clevios_P_coating_guide_08-03-18jb2.pdf] indicating there is no phase segregation in the absence of silica.

These results indicate PEDOT:PSS has undergone phase segregation forming a PEDOT rich surface to the PEDOT-PSS film likely due to hydrogen bonding between the sulfonate anions on PSS and the hydroxyl rich surface of silica. [S.-J. Wang, Y.-J. Choi, S. C. Gong, Y.-H. Kim, H.-H. Park, Mol. Cryst. Liq. Cryst. 2012, 568, 179] The phase segregation was not significant enough to induce high level ordering or crystal growth as the microstructure mostly remained amorphous based on glancing angle X-ray measurements. This could likely be due to PSS as previous reports did not observe PEDOT crystalline formation until it was removed. [Wang, 2012; N. Kim, S. Kee, S. H. Lee, B. H. Lee, Y. H. Kahng, Y.-R. Jo, B.-J. Kim, K. Lee, Adv. Mater. 2014, 26, 2268; D. Alemu, H.-Y. Wei, K.-C. Ho, C.-W. Chu, Energy Environ. Sci. 2012, 5, 9662] Since inter and intra-charge hopping is believed to be the dominant conduction mechanism in conducting polymers [A. Aleshin, R. Kiebooms, R. Menon, A. J. Heeger, Synth. Met. 1997, 90, 61] hydrogen bonding interactions leading to phase segregation between PEDOT and PSS enables more interchain interaction between the conducting PEDOT domains. Hence, the energy barrier for charge hopping is lowered leading to better charge transfer among the PEDOT chains. [D. Alemu, H.-Y. Wei, K.-C. Ho, C.-W. Chu, Energy Environ. Sci. 2012, 5, 9662]

### Example 14. Washability Studies

Sample Preparation: PEDOT-PSS (Conductive Polymer) was coated onto nonwoven PET synthetic leather (having 3%±1% surface area silica nanoparticles according to XPS) of 1 mm thickness using different techniques such screen printing, roller printing and brush printing. The sample textile size for coating was 1″×1″. 1 mm width line was laid onto the center of the fabric and then dried at 110° C. for 1 hour. The weight of the sample before and after the coating was measured using a microbalance to give the wet mass of PEDOT-PSS on the fabric. Water content in the printing solution was calculated from TGA which was used to calculate the weight of dry PEDOT-PSS on fabric. The resistance of the samples was measured using 4 line probe technique immediately after drying.

Washing durability: Step 1. Commercially available TIDE laundry detergent commercially available from Procter & Gamble was used for studying the washability of the fabric. 1% w/w solution of TIDE in DI water was prepared. Separate solution of DI water was used as control. Step 2. For hydrophobic treated fabrics, both sides of the fabric were sprayed with Scotchgard™ Fabric & Upholstery Protector (commercially available from 3M) and allowed to dry at room temperature for 6 hours. Step 3. The fabrics were stirred in these solutions (50 ml) to simulate agitation of the fabric for 5 min. The fabrics were then dried at 60° C. overnight. The resistances of the fabric before and after the wash were measured. Step 4. The sheet resistance was measured using a four line probe method before and after washing for samples without hydrophobic treatment. For the hydrophobic treated fabrics, both ends of the conductive wires were cut after each wash. Silver paste was applied to the ends followed by annealing at 60° C. for 30 min in order to make connections to the power source. The resistance was measured and the sheet resistance was calculated based on the dimension of the wires. Scotchgard™ was applied again following the same procedure in Step 2 before the next washing cycle.

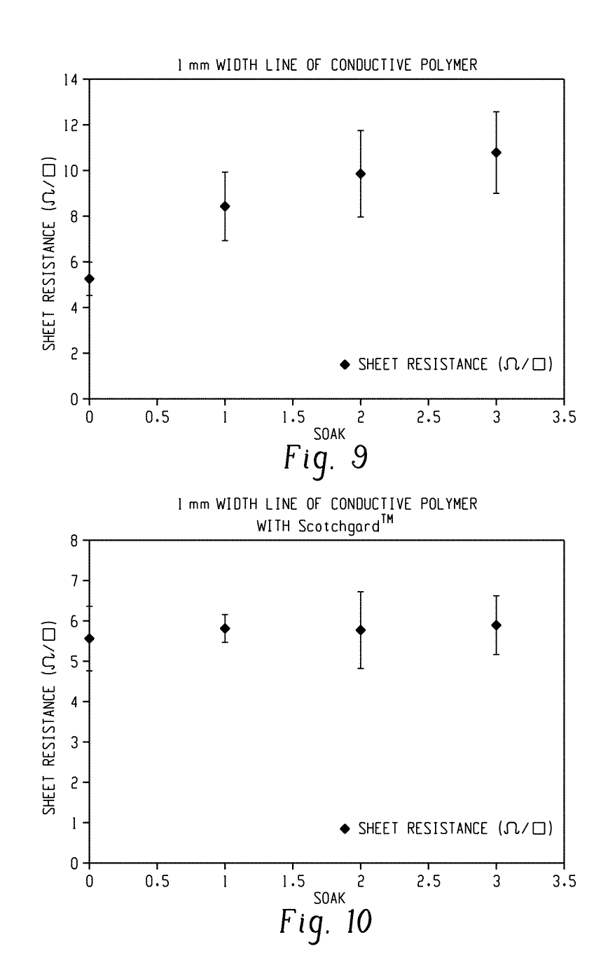

FIG. 9 illustrates the sheet resistance (ohm/□) of samples having a 1 mm width line of Conductive Polymer, where the samples have undergone a washing treatment (“Soak” on the x-axis). FIG. 10 illustrates the sheet resistance (ohm/□) of samples having a 1 mm width line of Conductive Polymer and Scotchgard™ treatment, where the samples have undergone a washing treatment (“Soak” on the x-axis). As shown by the results, the samples were washable, i.e., the electrical conductivity of the sample was substantially retained after the washing treatment. Such results are surprising as it was not previously known that a salt-polymer, e.g. a conductive polymer consisting of positively charged polythiophene and a negatively charged polystyrene sulfonate (PEDOT-PSS), would be able to withstand a wash cycle in the presence of laundry detergent, or that it could be stabilized using Scotchgard™. Rather, it would have been expected that a charged species such as PEDOT-PSS would either dissipate or there would be ion exchange with the surfactant of the laundry detergent to diminish electrical properties.

Although Example 14 was conducted using nonwoven PET synthetic leather, the stretchable electrically conductive structure exhibits similar washability.

### Example 15. PEDOT:PSS Screen Printed on Commercially Finished Spandex for ECG Monitoring Up to 180 Beats Per Minute (Bpm)

PEDOT:PSS is known to be a mixed conductor capable of carrying ionic as well as electronic current. While PEDOT is responsible for electronic conductivity, PSS contributes to ionic conductivity both of which aid in the transduction of ECG signal. Typically, commercial ECG electrodes consist of an Ag/AgCl electrode, a hydrogel consisting of an electrolyte, and an adhesive that helps in establishing skin contact. In this example, a conductive, compression t-shirt for monitoring ECG signals using commercially available materials and screen printing technique was prepared without the use of a hydrogel or an adhesive around the electrode. Screen printed PEDOT:PSS electrodes on a compression t-shirt present an interesting material for monitoring the cardiovascular activity of an athlete, not only during sedentary conditions but also during exercise. This example shows that PEDOT:PSS electrodes were able to record ECG signal in dry conditions due to their ability to function as both ionic and electronic conductors.

Materials: PEDOT:PSS-CLEVIOS PH1000 was obtained in a colloidal form (with a solid content of 1.25 wt %) from Heraeus. Triton™ X-100 (t-octylphenoxypolyethoxyethanol; t-Oct-C₆H₄—(OCH₂CH₂)×OH, x=9-10) and Dimethyl Sulfoxide (DMSO) were obtained from Sigma Aldrich and used without any further purification. Ag/AgCl snap buttons for ECG measurements were obtained from commercial sources, and nonwoven polyethylene terephthalate was obtained from Jo-Ann Fabrics. Ag/AgCl chloride was obtained from ConMed Corporation, U.S.A. A Speedball™ screen printing setup was obtained from Jo-Ann Fabrics. Cetaphil® moisturizing cream and Spandex compression shirts were obtained from local stores.

Methods: A formulation containing 93.5% CLEVIOS PH1000, 5% DMSO and 1.5% Triton™ X-100 was prepared and concentrated to 40% of its original weight by evaporating water at 60° C. for 6 hours. The solid content of the formulation was measured using thermogravimetric analysis (TGA). TGA was conducted on a TA Instruments thermogravimetric analysis Q500 by heating 10° C./min from room temperature to 110° C. in nitrogen, held isothermally at 110° C. for 1 hour, followed by heating at 10° C./min from 110° C. from 200° C. Screen printing was carried out using a Speedball™ screen with a nylon mesh of mesh count 110, and the squeegee was held at 45° using a custom-made holder. The printing speed was approximately 50 mm/sec. Several 1 mm by 25 mm wires were screen printed using the concentrated solution to study the effect of layer thickness on sheet resistance. To demonstrate the current carrying ability of the electrodes, various PEDOT:PSS wires of width 1 mm and length 10 cm were printed on the fabric.

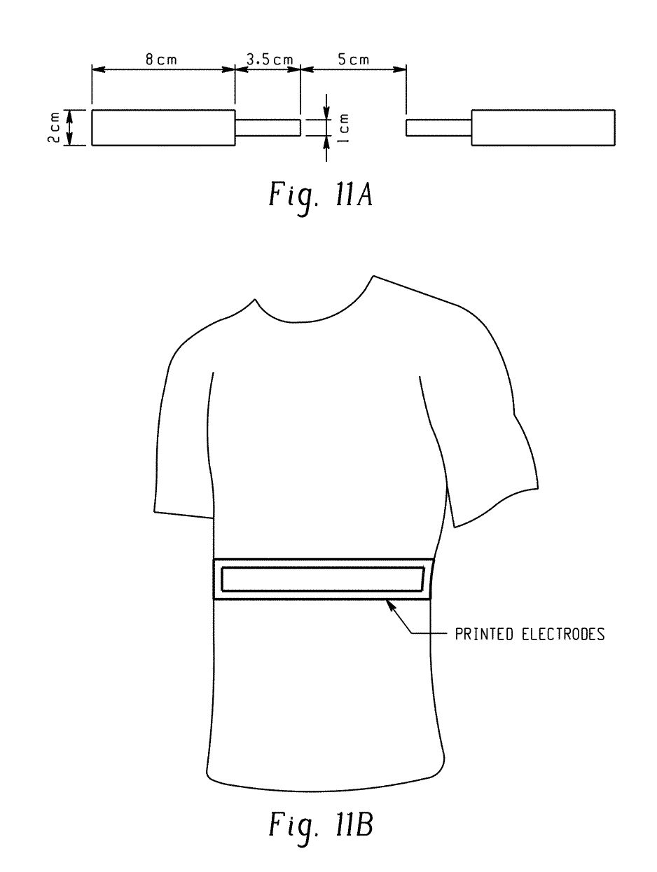

To make ECG electrodes, the concentrated formulation was printed onto 8 cm by 5 cm fabric, and 2 cm by 5 cm fabrics swatches were cut and connected to Ag/AgCl snap buttons for input to ECG amplifier. Fabric electrodes and Ag/AgCl were connected with PEDOT:PSS wires and subsequently used for measuring ECG signal. To demonstrate practical usage during exercise, the ECG electrodes were printed onto a commercially available compression t-shirt (FIG. 11B) with 84% polyester content. The design and dimensions of the ECG electrodes on the compression t-shirt is shown in FIG. 11A. The fabrics were first allowed to dry in air for 30 mins at 25° C. and then annealed in an oven at 110° C. for one hour.

Electrical characterization: The sheet resistance of the PEDOT:PSS coated textile sample was measured using a 4-line setup consisting of a Keithley 224 programmable source (Imax=101×10-3 A), a Keithley 2700 multimeter, and a custom made four-line probe cell. The sheet resistance was calculated using the equation: R s=R w/l, where R is the resistance obtained from slope of the I-V curve, w is the width of the sample, and 1 is the distance between the electrodes. Skin contact impedance was measured using a Hioki IM3570 (Hioki E.E. Corp, Nagano, Japan) with electrodes placed on the chest. The impedance spectra of completely soaked PEDOT:PSS fabric was recorded using a Gamry Potentiostat in the frequency range of 100 mHz-1 MHz, starting with the highest frequency.

Electrochemical Impedance Spectroscopy (EIS) of PEDOT:PSS coated fabric in dry and wet conditions, Procedure: A fabric swatch of dimensions 2 cm×2 cm was soaked with 0.25 g PEDOT:PSS formulation containing 93.5% PH 1000, 5% DMSO and 1.5% Triton X-100. The swatch was allowed to stand for 30 mins and then annealed at 110° C. for 1 hour. The dried swatch was sandwiched between two circular electrodes, which were then connected to a Gamry Reference 600 Potentiostat for EIS measurements with an amplitude of 5 mV in the frequency range of 100 mHz to 1 MHz. For measuring impedance of wet electrodes, PEDOT:PSS coated swatches were wet with water and excess of water was dabbed off using a wipe. Impedance Spectroscopy of Ag/AgCl electrode was done with the electrode attached to a stainless steel shim stock.

Scanning Electron Microscopy (SEM): The cross section of Screen printed PEDOT:PSS wires having a width of 1 mm was characterized using field emission scanning electron microscopy (FESEM, JEOL JSM-6335F). The sample was submerged in liquid nitrogen, and a blade was used to cut the cross-section.

Protocol for ECG measurement: Three healthy male subjects were recruited for this study. Oral consent was obtained from all the subjects. An ECG monitoring device was fabricated for measuring single lead ECG signals. Each ECG circuit obtained a Lead I signal from the chest via two electrodes with a virtual right-leg driven circuit. One subject was recruited to test the electrodes printed on the compression t-shirt.

ECG instrumentation and Signal Processing: The single lead ECG device used for Screen printed PEDOT:PSS electrodes provided a 3-dB cutoff from 0.5 to 150 Hz with the use of a second-order band-pass filter, and a sampling rate of 300 Hz. The filtered analog ECG signals were converted to digital data by using a 12-bit analog-to-digital converter (ADC) embedded in a micro-controller (MSP430F2618, Texas Instruments, TX, USA). A 6-point moving average notch filter was applied to the ECG signals for 60 Hz power noise rejection. The ECG signals were transmitted to a personal computer via Bluetooth wireless communication. A LabVIEW software (National Instruments, TX, USA) graphic user interface software was developed for real-time display and data storage for further off-line data analysis. Ag/AgCl snap buttons were used to connect leads to the ECG device, and an elastic chest strap was used to immobilize the electrodes onto a subject's chest. For fabric based electrodes, approximately 0.5 g of Cetaphil® lotion was rubbed onto the area where the electrodes were going to be placed. The lotion was used to reduce contact impedance for fabric electrodes due to the prevalence of dry skin. A chest strap was placed with one electrode on the left and the other electrode on the right side of the rib cage. Each experiment lasted 1 minute in the sitting position and subjects were instructed to remain relaxed during each experiment. A 30-second ECG segment containing stable data was chosen for analysis. First, the acquired ECG data was filtered with a 4th-order Butterworth bandpass filter with the cutoff bandwidth between 0.1 to 40 Hz. A non-local mean filtering algorithm as a secondary filtering step was applied offline to minimize the high frequency noise observed in the collected data. For measuring the ECG during exercising one participant was recruited to examine its performance. The experimental protocol included 51-resting (5 min), S2-walking (2 mph, 5 min), S3-fast-walking(3 mph, 5 min), S4-walking/running (4 mph, 5 min), S5-running (5 mph, 5 min), and S6-recovery (2 mph, 5 min).

Signal Processing: After filtering, R-wave peak detection was performed on the selected ECG segments using a robust QRS complex detection algorithm. ECG templates were computed for each selected ECG segment by creating an ensemble matrix with the corresponding ECG cycles aligned with respect to their R-peak locations and then averaged at each time instant. ECG amplitude was calculated using peak-to-peak values in each ECG template. The amplitude of the ECG signal from the fabric electrodes was then compared to commercially available Ag/AgCl.

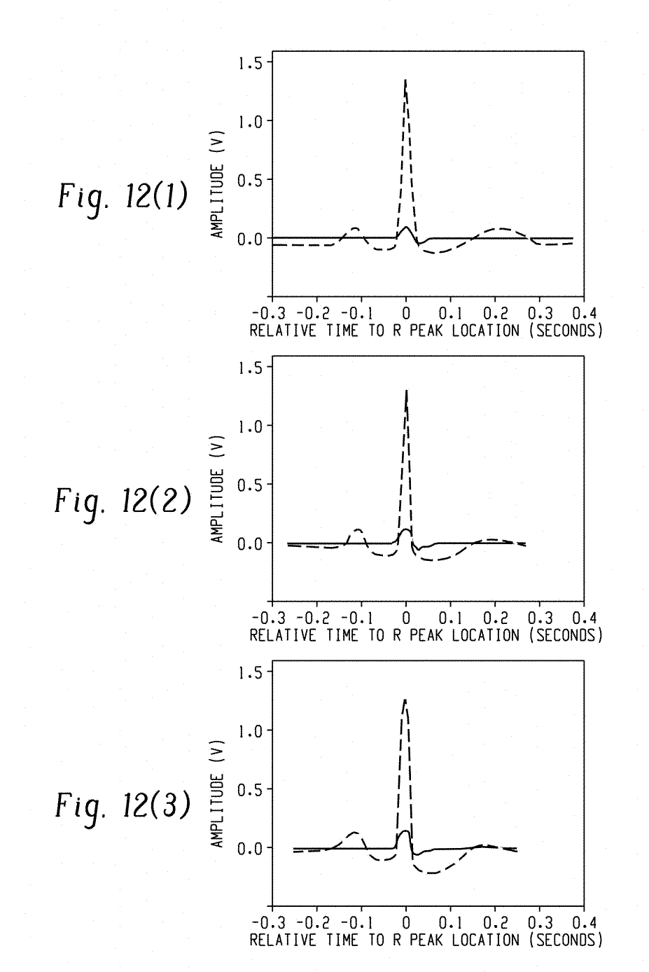

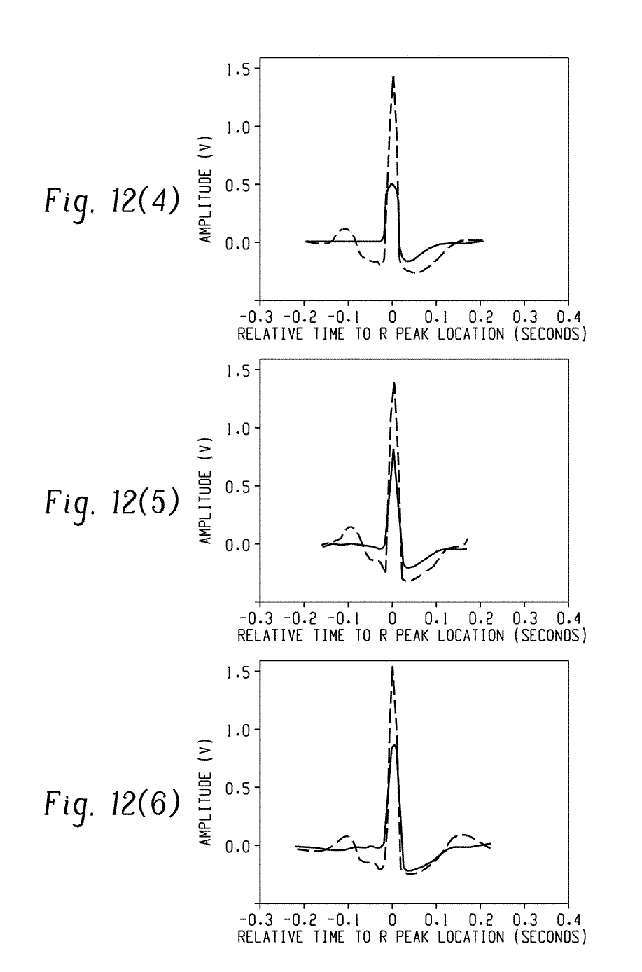

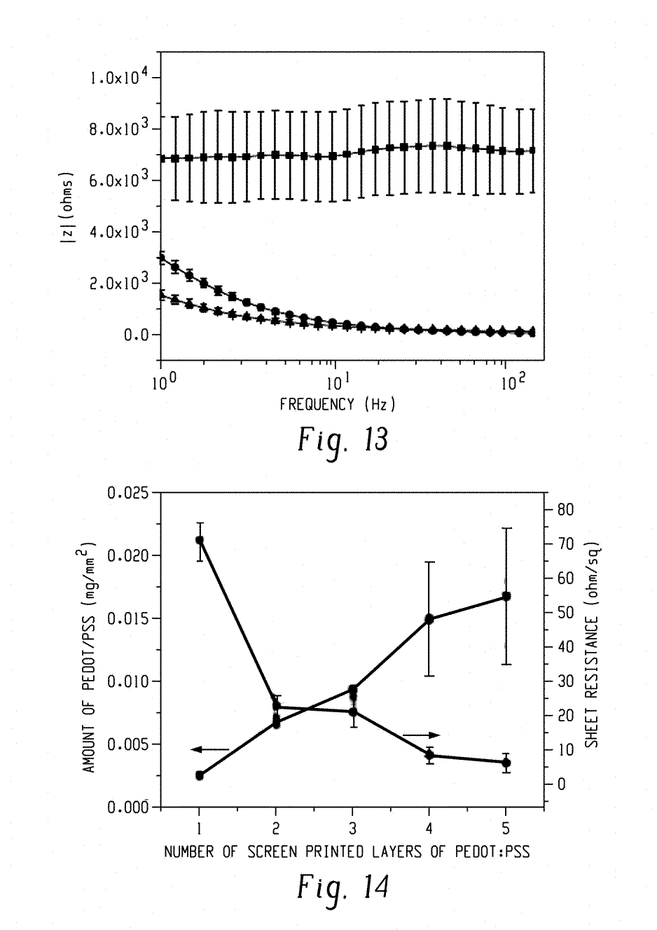

One subject was recruited to test the viability of the electrode for measuring ECG signal during exercise on a treadmill. An elastic chest strap was used to enhance skin contact with the electrode. Ag/AgCl electrodes were used as a control to simultaneously measure ECG along with printed PEDOT:PSS electrodes. FIG. 12 shows the ECG signal obtained from a subject under various exercising conditions: ECG waveform obtained from PEDOT:PSS electrodes on t-shirt (-) and Ag/AgCl electrode ( - - - ) under different running speeds: FIG. 12(1) 80 bpm, FIG. 12(2) 110 bpm, FIG. 12(3) 140 bpm, FIG. 12(4) 150 bpm, FIG. 12(5) 180 bpm, and FIG. 12(6) 120 bpm. A discernable ECG signal was obtained with printed electrodes under dry skin conditions during the start of the exercise. The amplitude of this signal increased almost 7-fold from S1-resting (5 min) to S5-running (5 mph, 5 min), which corresponds to an increase in the level of physical activity. When the speed was lowered to 2 mph, and the subject was in recovery, the amplitude of the ECG signal remained the same. The transpiration of water vapor from the skin during exercise leading to decrease in skin contact impedance could be attributed to an increase in the amplitude of the signal. This observation is further substantiated by the results obtained from the electrochemical impedance spectroscopy, which shows that the electrode impedance of PEDOT:PSS fabric drops when the fabric is fully soaked in water (FIG. 13 PEDOT:PSS electrode in Dry Condition (), PEDOT:PSS electrode in wet condition (▪), Ag/AgCl with Hydrogel (▴)). This reduced contact impedance existed even after completion of exercise because of hydrophilicity of PSS in PEDOT:PSS. The cross-correlation indices of ECG templates between the fabricated sensors and Ag/AgCl electrodes was approximately 83%, even in S4-walking/running (4 mph, 5 min) and S5-running (5 mph, 5 min), which there are high motion artifacts. The average heart rate was calculated by measuring the R—R interval and was found to be as high as 180 bpm, which corresponds to heart rate during High Intensity Interval Training (HIIT). HIIT is generally done at a heart rate that is 85%-95% of maximum heart rate (HRmax) which is calculated as HRmax=207−(0.7*age). The recorded value of 180 bpm reaches the value of maximum heart rate for a 15 year old undergoing HIIT and therefore the application of conductive, compression t-shirt can be extended to almost any age group above 15 years.

To demonstrate reusability of the conductive, compression t-shirt, a preliminary study was done with washing the t-shirt with commercially available detergent in a regular washing machine, and ECG signal was recorded at a heart rate of 170 bpm. Thus, PEDOT:PSS based fabric electrodes are a good candidate for measuring an ECG and that applications of such electrodes could also be extended to the realms of gaming and military in addition to sports medicine.

### Example 16

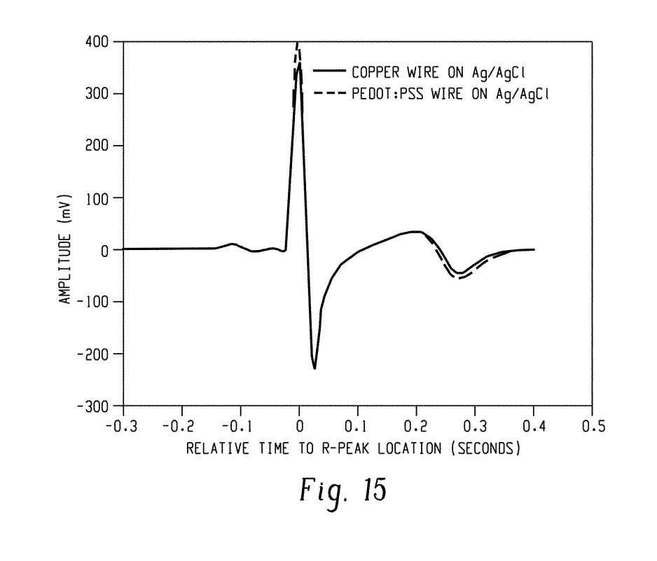

To understand the material aspects of the wearable ECG sensor, a systematic study was carried out to replace components of a typical ECG setup, which currently includes copper wires and Ag/AgCl electrodes. An optimization study was done on screen printing PEDOT:PSS on nonwoven polyethylene terephthalate (PET) fabrics. PEDOT:PSS coated textile was first demonstrated to function as a wire of 1 mm thickness with Ag/AgCl as an ECG electrode. To evaluate the change in resistance versus the amount of PEDOT:PSS on the fabric, a maximum of five layers of 2.5 cm length and 1 mm width were printed onto PET nonwoven textile. FIG. 14 shows the change in sheet resistance of the fabric as a function of wire thickness. Approximately 45 mg of 5 layers of PEDOT:PSS over an area of 25 mm2 gave a sheet resistance of 5.6 Ω/sq. To demonstrate the practical usage of the printed wires, a wire of length 10 cm and width 1 mm was printed onto PET. Three coatings of PEDOT:PSS were applied, and the overall resistance of the printed wires was measured to be ca. 3 kΩ This wire was then connected to an Ag/AgCl electrode attached to the chest, and an ECG signal was recorded which is shown in FIG. 15. The change to PEDOT:PSS wires resulted in a loss of only ca. 2% in the amplitude of ECG signal when compared to Ag/AgCl electrodes with copper wires, although the resistance of the wire was approximately three-fold higher than that of copper wire.

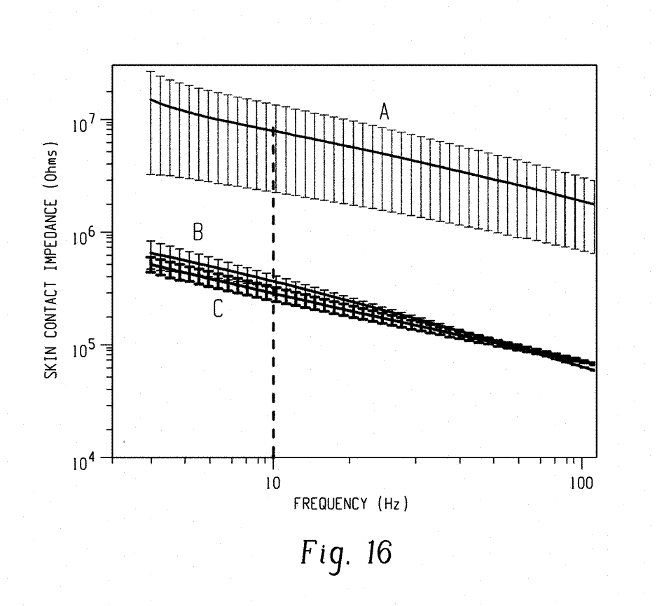

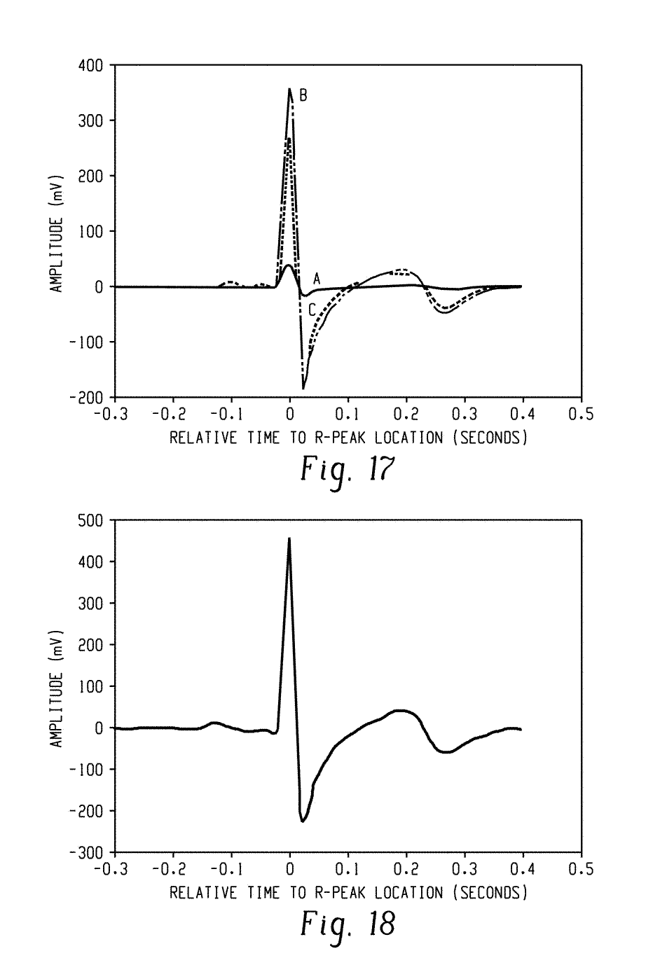

Screen printed PEDOT:PSS fabrics were then as electrodes for ECG application. The ECG signals collected from a screen printed PEDOT:PSS fabric that has an average resistance of ca. 5 Ω/sq were then compared with that of the commercially available Ag/AgCl electrode. To reduce the skin-electrode impedance, approximately 0.5 g of Cetaphil® lotion was applied onto the skin as opposed to coating the electrode with a conducting gel. The skin contact impedances of electrodes at 10 Hz, the frequency that corresponds to the QRS complex, were measured to be 0.35+0.04 MΩ and 0.27+0.6 MΩ for Ag/AgCl and screen printed PEDOT:PSS electrodes with lotion, respectively. This decrease in contact impedance substantially affected the amplitude of the ECG signal with screen printed electrodes, showing a 55.4% increase in amplitude compared to Ag/AgCl. FIG. 16 shows the variation of skin contact impedance with frequency. Screen printed PEDOT:PSS electrodes with underlying lotion on the skin have similar contact impedances to Ag/AgCl over a frequency range of 4-150 Hz. It is, therefore, clear that lowering the skin contact impedance plays a role in ECG recording, though a discernable ECG signal was still recorded with fabric PEDOT:PSS electrodes without any lotion on the skin, i.e. dry conditions. The contact impedance at 10 Hz was marginally lower for PEDOT:PSS electrodes with lotion on the skin than with dry electrodes, which further translates to variations in amplitude of ECG signal as shown in FIG. 17.

The complications in ECG signals are caused by unwanted motion artifacts and noise. While the motion artifacts of a Ag/AgCl electrode has been overcome by adhesive around the electrode, the motion artifacts of PEDOT:PSS based fabric electrodes has not been explored fully. In this study, the conformity of the compression shirt onto the skin ensured that electrodes stayed in place during measurements which were further reinforced by a chest strap. The thermal noise generated by the inherent resistive characteristic of PEDOT:PSS is proportional to square root of its resistance for a given bandwidth. The signal-to-noise ratio (SNR) of the PEDOT:PSS fabrics with a sheet resistance of 5 ohm/sq was found to be 15.42 dB in dry skin conditions which increased to 29.59 dB upon application of lotion on skin. Earlier groups have found the SNR to be 11.01 dB for three layers of inkjet printed PEDOT:PSS on paper. This result points out how the resistance (thermal noise) of the electrode together with skin contact impedance influences the signal quality. The signal becomes noisy with an increase in resistance of the electrode. Inkjet printing results in less amount of PEDOT:PSS deposited which leads to lower sheet resistance and lower signal-to-noise ratio. Additionally, the resistance of PEDOT:PSS coated fabric is not affected by temperature above 0° C. until 100° C. as shown by earlier work where PEDOT:PSS coated textile with resistance ca. 2Ω exhibited metallic character at temperatures greater than 0° C. is reported.

The electrodes used in this study have a sheet resistance of ca.5 Ω/sq and therefore, can be used measured ECG signal in real time conditions where the temperature could vary between 0° C. to 50° C. Screen printing techniques combined with surface chemistry of fabrics can be exploited to coat PEDOT:PSS with low sheet resistances while retaining the metallic character. Also, the method of screen printing is already established in the industry for making organic electronic devices, which would allow for scalability in the production of ECG electrodes

Up until this stage, PEDOT:PSS was demonstrated as both wires and ECG electrodes. PEDOT:PSS electrodes were connected with PEDOT:PSS wires via snap buttons for recording ECG signals. Drawing on conclusions from the initial ECG testing, the skin was prepared by applying 0.5 g lotion over an area on skin with length 40 cm and width 6 cm. The ECG waveform obtained (shown in FIG. 18) demonstrated PQRST complex. The amplitude of the signal obtained was found to be comparable with that of Ag/AgCl. This result supports the concept of using organic materials integrated into wearables with the possible replacement of metals as both wires and electrodes.

This example describes the fabrication of an all organic ECG electrode using PEDOT:PSS. Exploiting the mixed nature of conduction, PEDOT:PSS based electrodes were shown to record ECG signal in dry condition. The formation of sulfo-silyl esters has been attributed to low sheet resistances of ca. 5 ohm/sq leading to signal loss of only 2% when PEDOT:PSS wires are used instead of copper wires. Screen Printed PEDOT:PSS electrodes on a compression t-shirt were shown to record ECG signal under physical activity at a heart rate of 180 bpm. Increased ionic conductivity brought about by transpiration of water results in an increase in the amplitude of the signal. PEDOT:PSS demonstrated the ease of processing in that it can be used as an organic wire as well as an electrode resulting in a signal comparable to Ag/AgCl electrode.

Nonwoven polyester fabric containing silica, e.g. as a delustering agent or desiccant, when fully soaked with PEDOT:PSS has been shown to have high current carrying capacities of ca. 10 A/mm2. Considering that every three units in PEDOT carries a positive charge, which is stabilized by one unit of negative charge on PSS, calculations suggest that approximately 4 out of every 5 units of styrene sulfonic acid in polystyrene sulfonate are unbound (Scheme 1.). These unbound groups are available to react with the fabric through a covalent bond between the silanol groups contained in the silica and the free sulfonic acid groups of polystyrene sulfonic acid (PSSH). The pKa value of sulfonic acid groups in PSS has been reported to be 1, while that of silanols in silica to be 6.8. Wet PEDOT:PSS on fabric will cause protonation of silanol groups, which, followed by condensation with sulfonic acid, leads to the formation of sulfo-silyl esters leading to the covalent bond formation with the fabric.

### Scheme 1. Calculations:

Basis: 1 gram of dry PEDOT:PSS in CLEVIOS PH-1000 (1:2.5 w/w) Assumptions: One in every three units of EDOT carries a positive charge Calculations:

$$Amount of PEDOT = \frac{1}{3.5} = 0.285 g$$

$$Amount of PSS = \frac{2.5}{3.5} = 0.714 g$$

$$Molecular weight of EDOT repeat = 140 g / mol$$

$$Molecular weight of styrene sulfonic acid ( SSA ) repeat = 180 g / mol$$

$$Number of units of EDOT = \frac{amount of EDOT}{Molecular weight of EDOT repeat} \times {N}_{A} = 2.3 \times {10}^{- 3} {N}_{A}$$

$$Number of units of ( SSA ) = \frac{amount of SSA}{Molecular weight of SSA repeat} \times {N}_{A} = 3.9 \times {10}^{- 3} {N}_{A}$$

$$Number of units of {EDOT}^{+} = \frac{Number of units of EDOT}{3} = 6.7 \times {10}^{- 4} {N}_{A}$$

$$Number of units required {SSA}^{-} = Number of units of {EDOT}^{+} = 6.7 \times {10}^{- 4} {N}_{A}$$

$$Excess SSA left = 3.9 \times {10}^{- 3} {N}_{A} - 6.7 \times {10}^{- 4} {N}_{A} = 3.26 \times {10}^{- 3} {N}_{A} \\ Ratio of unbound SSA to total SSA = \frac{3.26 \times {10}^{- 3} {N}_{A}}{3.9 \times {10}^{- 3} {N}_{A}} = \frac{0.83}{1} ≅ \frac{4}{5}$$

The singular forms “a,” “an,” and “the” include plural referents unless the context clearly dictates otherwise. The endpoints of all ranges directed to the same characteristic or component are independently combinable and inclusive of the recited endpoint.

While the invention has been described in detail in connection with only a limited number of embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Additionally, while various embodiments of the invention have been described, it is to be understood that aspects of the invention can include only some of the described embodiments. Accordingly, the invention is not to be seen as limited by the foregoing description, but is only limited by the scope of the appended claims.

What is claimed is:

1. A bio-potential wearable monitoring system, comprising:
an electrode comprising a conductive polymer fabric, and
a wiring element in electrical communication with the electrode;
wherein the conductive polymer fabric is
a.) an electrically conductive fibrous substrate comprising:
a fibrous substrate comprising polymeric fibers comprising nucleophile derivatized nanoparticles wherein a portion of the nucleophile derivatized nanoparticles are located at the surface of the polymeric fiber; and
an electrically conductive polymer film disposed on at least a portion of the polymeric fibers of the fibrous substrate and at least in partial contact with the nucleophile derivatized nanoparticles; or
b.) a stretchable electrically conductive structure comprising
a stretchable insulating substrate comprising nucleophile derivatized nanoparticles located at the surface of the stretchable insulating substrate, wherein the stretchable insulating substrate is a fiber or fabric; and
a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate.

2. The monitoring system of claim 1, wherein the wearable monitoring system is in the form of a garment, footwear, headwear, wrist band, chest strap, or belt.

3. The monitoring system of claim 1 or 2, wherein the monitoring system is an electrocardiogram system, an electroencephalogram system, an electromyography system, heart rate system, respiratory rate system, bioelectrical impedance analysis system, or an electrodermal activity system.

4. The monitoring system of claim 1 or 2, wherein the monitoring system is an electrocardiogram system
i) having a signal to noise ratio range of 3 to 30 dB; and
ii) capable of measuring ECG in the heart rate range of 40 to 180 bpm while in the ‘wet’ (with lotion and/or hydrogel) condition.

5. The monitoring system of claim 1 or 2, wherein the monitoring system is an electrocardiogram system
i) having a signal to noise ratio range of 3 to 30 dB; and
ii) capable of measuring ECG in the heart rate range of 40 to 180 bpm while in the ‘dry’ (without lotion or hydrogel) condition.

6. The monitoring system of any one of the preceding claims, wherein the nucleophile derivatized nanoparticles of a.) electrically conductive fibrous substrate and b.) stretchable electrically conductive structure comprise silica, titania, alumina, calcium oxide, amine functionalized nanoparticles, or a combination thereof.

7. The monitoring system of any one of the preceding claims, wherein
the electrically conductive polymer of the a.) electrically conductive fibrous substrate is PEDOT:PSS, a poly(3,4-ethylenedioxythiophene), a substituted poly(3,4-ethylenedioxythiophene), poly(thiophene), a substituted poly(thiophene), poly(pyrrole), a substituted poly(pyrrole), poly(aniline), a substituted poly(aniline), poly(acetylene), poly(p-phenylenevinylene) (PPV), a poly(indole), a substituted poly(indole), a poly(carbazole), a substituted poly(carbazole), a poly(azepine), a (poly)thieno[3,4-b]thiophene, a substituted poly(thieno[3,4-b]thiophene), a poly(dithieno[3,4-b:3′,4′-d]thiophene), a poly(thieno[3,4-b]furan), a substituted poly(thieno[3,4-b]furan), a derivative thereof; and
b.) the conducting polymer of the conducting polymer:template polymer comprises units of a conducting monomer wherein the conducting monomer is thiophene, substituted thiophene, 3,4-ethylenedioxythiophene, thieno[3,4-b]thiophene, substituted thieno[3,4-b]thiophene, dithieno[3,4-b:3′,4′-d]thiophene, thieno[3,4-b]furan, substituted thieno[3,4-b]furan, bithiophene, substituted bithiophene, pyrrole, substituted pyrrole, phenylene, substituted phenylene, naphthalene, substituted naphthalene, biphenyl and terphenyl and their substituted versions, phenylene vinylene, substituted phenylene vinylene, aniline, substituted aniline, the monomers disclosed herein as structures (I)-(XXIX), or a combination thereof; and
the template polymer is a polyanion acting as a counterion for a conducting polymer.

8. The monitoring system of any one of the preceding claims, wherein
the electrically conductive polymer of the a.) electrically conductive fibrous substrate is PEDOT:PSS; and
b.) the conducting polymer:template polymer is PEDOT:PSS.

9. The monitoring system of any one of the preceding claims, wherein
the polymeric fiber of the a.) electrically conductive fibrous substrate is nylon 6, nylon 66, nylon 610, nylon 12, co-polymerized nylon, polyethylene terephthalate, polytrimethylene terephthalate, polybutylene terephthalate, polypropylene terephthalate, polyurethane, polypropylene, polyethylene, spandex (polyurethane-polyurea copolymer), polyester-based polyurethane, copolymers thereof, or a combination thereof; and
the stretchable insulating substrate of the b.) stretchable electrically conductive structure comprises a polyester-polyurethane copolymer and optionally further comprises polyacrylic, polyamide, polycarbonate, polyether, polyester, polyethylene, polyimide, polyurethane, polyurea, polythiourea, polysiloxane, polyisoprene, polybutadiene, polyethylene oxide, polylactic acid, blends or copolymers thereof.

10. The monitoring system of any one of the preceding claims, wherein the conductive organic particle of the b.) stretchable electrically conductive structure is graphene, graphite, a combination of graphene and graphite, carbon nanotubes, buckyballs, “n-type” small molecules, or a combination thereof.

11. The monitoring system of any one of the preceding claims, wherein the wiring element is a metal wire (e.g. copper, silver, etc.), or a conductive polymer coated fabric.

12. The monitoring system of any one of the preceding claims, wherein the wearable monitoring system receives bio-potential signals that can be recorded and tracked by a fixed computer or a mobile device using wireless communication.

13. A method of measuring a bio-potential using the monitoring system of any one of the preceding claims.

14. The method of claim 13, wherein the bio-potential is measured in dry conditions.

15. The method of claim 13, wherein the bio-potential is measured in wet conditions.

16. A method of making a bio-potential wearable monitoring system, comprising:
forming an electrode comprising a conductive polymer fabric, and
connecting a wiring element to the electrode, wherein the wiring element is in electrical communication with the electrode; and
wherein the conductive polymer fabric is
a.) an electrically conductive fibrous substrate comprising:
a fibrous substrate comprising polymeric fibers comprising nucleophile derivatized nanoparticles wherein a portion of the nucleophile derivatized nanoparticles are located at the surface of the polymeric fiber; and
an electrically conductive polymer film disposed on at least a portion of the polymeric fibers of the fibrous substrate and at least in partial contact with the nucleophile derivatized nanoparticles; or
b.) a stretchable electrically conductive structure comprising
a stretchable insulating substrate comprising nucleophile derivatized nanoparticles located at the surface of the stretchable insulating substrate, wherein the stretchable insulating substrate is a fiber or fabric; and
a conducting polymer:template polymer coating disposed on at least a portion of a surface of the stretchable insulating substrate through which a chemical bond forms between at least one anion of the template polymer and nucleophile derivatized nanoparticles located at the surface of the stretchable insulating substrate.

17. The method of claim 16, wherein the method of making the a.) electrically conductive fibrous substrate comprises disposing an electrically conductive polymer onto the fibrous substrate;
optionally surface treating the fibrous substrate prior to the disposing step, wherein the surface treating is a plasma treatment, a solvent treatment, or a combination thereof.

18. The method of claim 17, wherein the electrically conductive polymer is drop cast, spray coated, ink jet coated, dip coated, spin coated, gravure coated, extrusion coated onto the fibrous substrate, or the fibrous substrate is soaked in a mixture of electrically conductive polymer and solvent.

19. The method of claim 17, wherein the method of making the b.) stretchable electrically conductive structure comprises
providing a stretchable insulating substrate comprising nucleophile derivatized nanoparticles located at the surface of the stretchable insulating substrate, wherein the stretchable insulating substrate is a fiber or fabric;
forming a conducting polymer:template polymer coating on at least a portion of a surface of the stretchable insulating substrate to form a stretchable electrically conductive structure,
optionally further wherein the stretchable insulating substrate is plasma treated before the forming the conducting polymer:template polymer coating.

20. The method of claim 19, wherein the conducting polymer:template polymer coating is formed using a casting process, tape casting, flow coating, spray coating, spin coating, ink jetting, dip coating, or a combination thereof.

21. The method of claim 19, further comprising, disposing a conductive organic particle on at least a portion of a surface of the stretchable insulating substrate.
